(19) United States
(12) Patent Application Publication
(10) Pub. No.: US 2025/0005717 A1
(43) Pub. Date: Jan. 2, 2025

(54) Methods and Systems for Enhancing Optical Textural Details of Retinal Features Relevant to a Health Condition

(71) Applicant: AIROTA DIAGNOSTICS LIMITED, Hong Kong (CN)

(72) Inventors: KAI-SHUN CHRISTOPHER LEUNG, Hong Kong (CN); KA-NGAI ALEXANDER LAM, Hong Kong (CN)

(21) Appl. No.: 18/755,338

(22) Filed: Jun. 26, 2024

Related U.S. Application Data
(60) Provisional application No. 63/510,747, filed on Jun. 28, 2023.

(51) Int. Cl. G06T 5/60; G06T 7/00

(52) U.S. Cl. CPC G06T 5/60; G06T 7/0012; G06T 2207/30041

(57) ABSTRACT

This application is directed to enhancing retinal images of a patient's eye and assessing a retina-related health condition. A computer system obtains a first visual representation of retinal nerve fiber bundles of a retina, and the first visual representation indicates optical textural details of the retinal nerve fiber bundles of the retina with a first level of detail. The computer system applies a detail enhancement model to the first visual representation. Based on applying the detail enhancement model to the first visual representation, the computer system generates a second visual representation of the retinal nerve fiber bundles of the retina. The second visual representation indicates the optical textural details with a second level of detail that is distinct from the first level of detail.

### RELATED APPLICATIONS

This application is a continuation of, and claims priority to, U.S. Provisional Patent Application No. 63/510,747, filed Jun. 28, 2023, entitled “Methods and systems for increasing optical textural details of axonal fiber bundles on Retinal-nerve-fiber-layer Optical Texture Analysis (ROTA) map,” which is incorporated by reference herein in its entirety.

### TECHNICAL FIELD

The present application generally relates to medical imaging technology, including methods and systems for enhancing optical textural details of retinal features relevant to a health condition in retinal images.

### BACKGROUND

Eye diseases such as glaucoma oftentimes cause visual field losses that adversely affect the quality of life of patients. Optical coherence tomography (OCT) provides an objective solution to determine structural integrity of optic nerve and macula of a human eye, while perimetry tests has typically been applied to detect actual functional deficits in the visual field of the eye.

Alternative processes have been introduced to mitigate the need for performing perimetry tests, or to increase the diagnostic accuracy resulting from such tests. Some of these alternative processes may include capturing cross-sectional images of a patient's retina to identify aspects of the patient's retinal nerve fiber bundles. However, similar to many medical imaging technologies, the diagnostic performance of such imaging process can be compromised in OCT scans with low image clarity (e.g., low scan resolution), obscuring the optical textural details of axonal fiber bundles and confounding the detection of retinal nerve fiber defects and their changes over time. Here, a method to increase the optical textural details of such imaging techniques is described.

### SUMMARY

Disclosed embodiments include systems and methods for enhancing optical textural details of retinal features. Some implementations of computer-implemented methods are applied to increase the optical textural details of retinal axonal fiber bundles on the retinal nerve fiber layer (RNFL) optical texture analysis (ROTA) map acquired from a patient's eye using a machine learning model. In some embodiments, thickness and reflectance data of the RNFL are extracted from a set of cross-sectional scans of the retina captured with an optical coherence tomography (OCT) imaging device. A ROTA map is derived from integration of the thickness and reflectance data of the RNFL, and used by the machine learning model to generate an enhanced ROTA map with increased optical textural details of retinal axonal fiber bundles. In some embodiments, the machine learning model includes a deep neural network, which is trained with a training dataset. The training dataset includes input ROTA maps having lower optical textural details of the axonal fiber bundles and associated output ROTA maps with higher optical textural details of the axonal fiber bundles. Each data pair includes an input ROTA map and an output ROTA map and corresponds to the same scanned region in the same eye. The trained machine learning model may reveal the axonal fiber bundles with additional optical textural details on the enhanced ROTA map, thereby improving the details and clarity of retinal axonal fiber bundles when the OCT imaging device is limited by scanning speed or resolution.

In one aspect, a method for enhancing optical textural details of retinal features relevant to a health condition is implemented at a computing system. The method includes obtaining a first visual representation of retinal nerve fiber bundles of a retina, the first visual representation indicating optical textural details of the retinal nerve fiber bundles of the retina with a first level of detail. The method further includes applying a detail enhancement model to the first visual representation. And the method further includes, based on applying the detail enhancement model to the first visual representation, generating a second visual representation of the retinal nerve fiber bundles of the retina, the second visual representation indicating the optical textural details with a second level of detail, distinct from the first level of detail. In some embodiments, the method further includes determining a health condition associated with the retina based on the second visual representation.

In some embodiments, each of the first level of detail and the second level of detail includes a respective image resolution.

In some embodiments, the method further includes training a neural network of the detail enhancement model using a plurality of paired data samples. Each paired data sample includes a first respective visual representation and a second respective visual representation corresponding to a respective ground truth for a respective retina.

In some embodiments, the detail enhancement model comprises a plurality of different machine learning models. The method further includes generating the second visual representation further includes applying each of the plurality of different machine learning models to process the first visual representation.

In some embodiments, the detail enhancement model includes a series of different machine learning models. Generating the second visual representation further includes applying the series of machine learning models successively. A first machine learning model is applied to process the first visual representation and generate a first intermediate representation, and a second machine learning model is applied to process the first intermediate representation and generate a second intermediate representation.

According to another aspect of the present application, a computer system includes one or more processing units, memory, and a plurality of programs stored in the memory. The programs, when executed by the one or more processing units, cause the computer system to perform any of the methods for enhancing optical textural details of retinal features as described above.

According to another aspect of the present application, a non-transitory computer readable storage medium stores a plurality of programs for execution by a computer system having one or more processing units. The programs, when executed by the one or more processing units, cause the computer system to perform any of the methods for enhancing optical textural details of retinal features as described above.

### BRIEF DESCRIPTION OF DRAWINGS

The accompanying drawings are included to provide a further understanding of the embodiments, are incorporated herein and constitute a part of the specification. The drawings illustrate the described embodiments and together with the description serve to explain the underlying principles.

FIGS. 1A to 1B are an example diagnostic evaluation platforms on which optical textural details of the retinal nerve fiber bundles on a ROTA map is enhanced and the enhanced ROTA map is applied to determine a health condition associated with the retina, in accordance with some embodiments.

FIG. 2 is an example data environment that facilitates communication and processing of retinal data, in accordance with some embodiments.

FIG. 3 is a block diagram of a computer system 300 configured to increase optical textural details of the retinal nerve fiber bundles on a ROTA map generated from a plurality of OCT scan images 108 of the retina, in accordance with some embodiments.

FIGS. 4A to 4C are example user interfaces showing visual representations including optical textural details of a retina, in accordance with some embodiments.

FIG. 5 depicts an example computer-implemented system for enhancing optical textural details of retinal features using a detail enhancement model, in accordance with some embodiments.

FIGS. 6A to 6E illustrate example training data used to train a detail enhancement model for enhancing optical textural details of retinal features, in accordance with some embodiments.

FIG. 6F depicts an example process for enhancing optical textural details of retinal features in an input ROTA map using a machine learning model with a U-Net architecture, in accordance with some embodiments.

FIGS. 7A to 7D are example processes of enhancing a detail level of a first visual representation of a retina with a detail enhancement model including a plurality of machine learning models, in accordance with some embodiments.

FIG. 8 is a flowchart of an example method for enhancing optical textural details of retinal features in accordance with some embodiments.

### DETAILED DESCRIPTION

Reference will now be made in detail to specific embodiments, examples of which are illustrated in the accompanying drawings. In the following detailed description, non-limiting specific details are set forth in order to assist in understanding the subject matter presented herein. It will be apparent to one of ordinary skill in the art that various alternatives may be used without departing from the scope of claims, and the subject matter may be practiced without these specific details. For example, it will be apparent to one of ordinary skill in the art that the subject matter presented herein can be implemented on many types of computer systems that support diagnostic evaluation and monitoring of eye diseases.

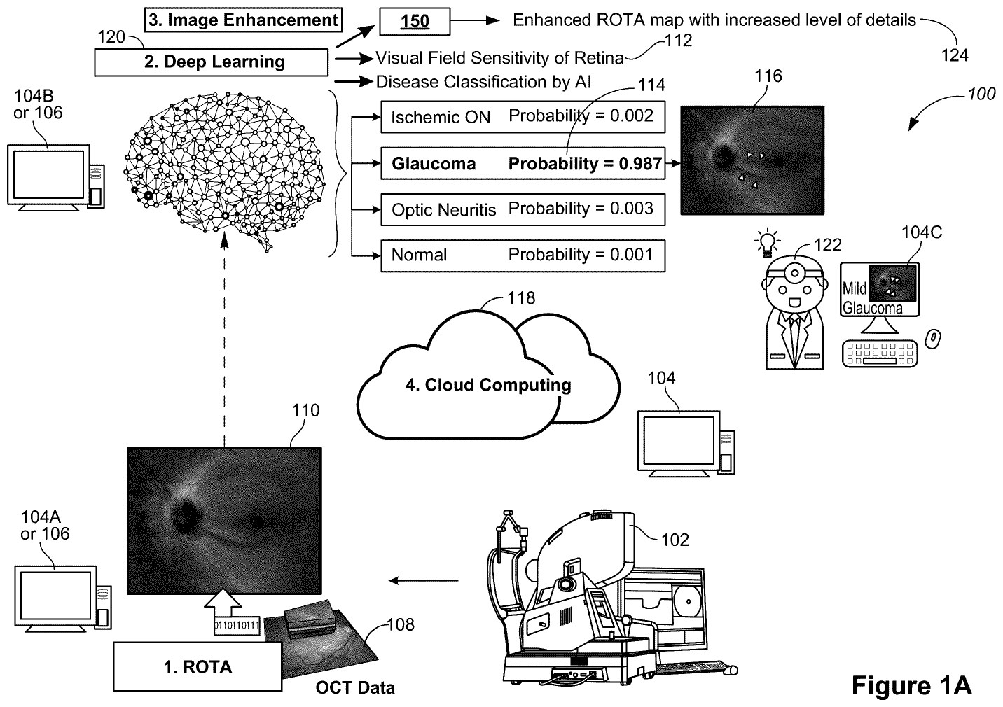

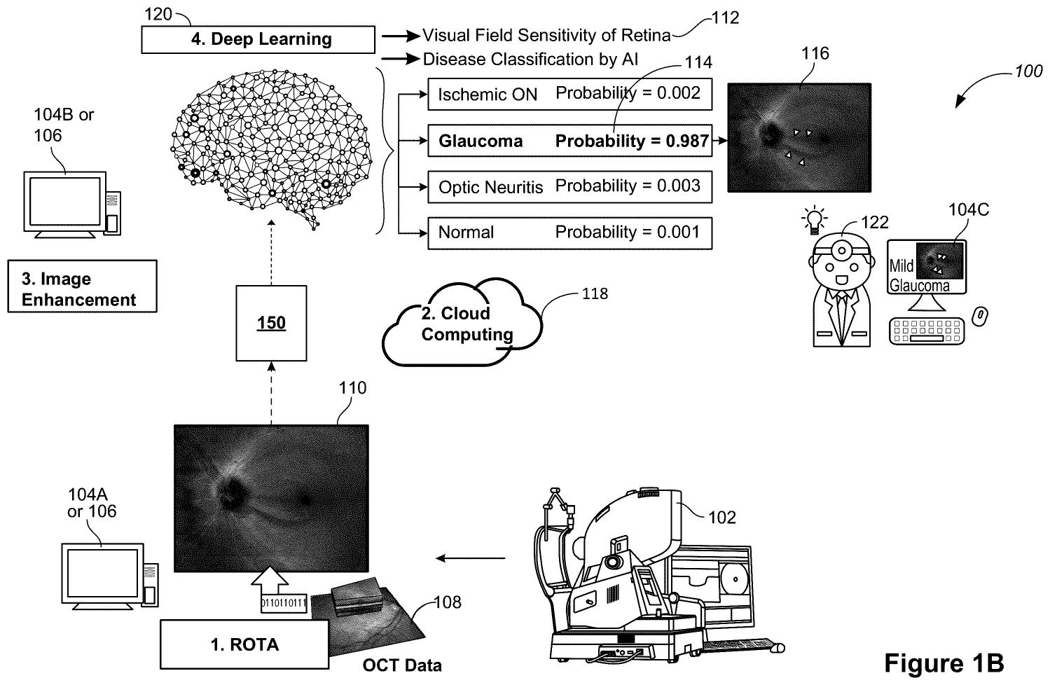

FIG. 1A is an example diagnostic evaluation platform 100 on which at least optical textural details of retinal nerve fiber bundles on a ROTA map 110 of a retina is increased, in accordance with some embodiments. The diagnostic evaluation platform 100 includes an optical coherence tomography (OCT) device 102 and one or more computer devices 104 (e.g., devices 104A and 104B). The OCT device 102 is configured to capture a plurality of cross-sectional scan images 108 of a retina including an inner retinal layer. A first computer device 104A is optionally distinct from the OCT device 102 or integrated in the OCT device 102. The first computer device 104A is configured to obtain the plurality of cross-sectional scan images 108 of the retina and generates a ROTA map 110 of the inner retinal layer from the plurality of cross-sectional scan images 108. The ROTA map 110 includes a plurality of pixels, and each pixel of the ROTA map corresponds to a respective optical texture signature value S providing information about tissue composition and optical density of the inner retinal layer at a respective retinal location. A second computer device 104B is optionally distinct from the first computer device 104A or includes the first computer device 104A. The second computer device 104B is configured to apply one or more machine learning models 120 to process the ROTA map 110 of the inner retinal layer to generate enhanced ROTA map 124 with increased level(s) of one or more details of the retinal nerve fiber bundles, determine visual field sensitivity 112 of the retina, estimate a probability 114 of each of one or more eye diseases, identify a defect location 116 in the RNFL, or implement other retinal analytic tasks. A third computer device 104C is optionally distinct from the first and second computer devices 104A and 104B or includes one or both of the first and second computer devices 104A and 104B. The third computer device 104C is configured to report the plurality of scan images 108, the ROTA map 110, the enhanced ROTA map 124 with increased optical textural details, the visual field sensitivity 112 of the retina, the probability 114 of each of one or more eye diseases, the defect location 116 in the RNFL, or any other retinal analytic results to a doctor 122 or to a patient.

FIG. 1B shows another example diagnostic evaluation platform 100 which further includes a server 106. In some embodiments, the server 106 is configured to receive the plurality of cross-sectional scan images 108, generate the ROTA map 110 of the inner retinal layer from the plurality of cross-sectional scan images 108, train/apply a detail enhancement model 150 to enhance a level of detail of the ROTA map 110, or train/apply the one or more machine learning models 120 to process the ROTA map 110 of the inner retinal layer to implement one or more retinal analytic tasks. In some embodiments, the first computer device 104A is coupled to the OCT device 102 locally at a venue, and generates the ROTA map 110 of the inner retinal layer from the plurality of cross-sectional scan images 108 captured locally by the OCT device 102. The ROTA map 110 is uploaded to the server 106 via one or more communication networks 118. The server 106 receives the ROTA map 110, applies the detail enhancement model 150 to enhance the level of detail of the ROTA map 110, and applies the one or more machine learning models 120 to process the enhanced ROTA map 124 to implement one or more retinal analytic tasks including determining the visual field sensitivity 112 of the retina, estimating a probability 114 of each of one or more eye diseases, identifying a defect location 116 in the RNFL, or other retinal analytic tasks. In some embodiments, the detail enhancement model 150 is stored at the server 106, and the ROTA map 110 is provided to the detail enhancement model 150 to generate the enhanced ROTA map 124 with enhanced optical textural details. Alternatively, in some embodiments, the plurality of cross-sectional scan images 108 are uploaded to the server 106 by the OCT device 102. The server 106 receives the plurality of cross-sectional scan images 108, generates the ROTA map 110 from the cross-sectional scan images 108, enhances the level of detail of the ROTA map 110, and implements one or more retinal analytic tasks (e.g., determining the visual field sensitivity 112 of the retina) using one or more machine learning models 120. The third computer device 104C downloads the retinal analytic results of the one or more retinal analytic tasks from the server 106, and presents the retinal analytic results to the doctor 122 or to the patient for review.

In some embodiments, the server 106 does not include any of the computer devices 104A-104C. The first and second computer devices 104A and 104B are optionally located at the same location with the OCT device 102 or the third computer device 104C. The server 106 is configured to train the detail enhancement model 150 and the one or more machine learning models 120 using training datasets and provide the trained machine learning models 120 and 150 to the second computer device 104B, allowing the second computer device 104B to process the ROTA map 110 and implement one or more retinal analytic tasks locally.

The OCT device 102, one or more computer devices 104, and the server 106 are communicatively coupled to each other via one or more communication networks 118, which are used to provide communications links among devices connected together within the diagnostic evaluation platform 100. The one or more communication networks 118 may include connections, such as a wired network, wireless communication links, or fiber optic cables. Examples of the one or more communication networks 118 include local area networks (LAN), wide area networks (WAN) such as the Internet, or a combination thereof. The one or more communication networks 118 are implemented using any known network protocol, including various wired or wireless protocols, such as Ethernet, Universal Serial Bus (USB), FIREWIRE, Long Term Evolution (LTE), Global System for Mobile Communications (GSM), Enhanced Data GSM Environment (EDGE), code division multiple access (CDMA), time division multiple access (TDMA), Bluetooth, Wi-Fi, voice over Internet Protocol (VOIP), Wi-MAX, or any other suitable communication protocol. A connection to the one or more communication networks 118 may be established either directly (e.g., using 3G/4G connectivity to a wireless carrier), or through a network interface (e.g., a router, switch, gateway, hub, or an intelligent, dedicated whole-home control node), or through any combination thereof. In some embodiments, the one or more communication networks 118 allow for communication using any suitable protocols, such as Transmission Control Protocol/Internet Protocol (TCP/IP).

In some embodiments, the platform 100 includes the detail enhancement model 150 at the computer device 104 (e.g., 104A, 104B, or 104C), and the detail enhancement model 150 employs one or more techniques discussed in more detail below for enhancing an image quality of the ROTA map 110. Stated another way, the computer system 104 includes the computer devices 104A, 104B, and 104C, which may be the same computer device, and the detail enhancement model 150 may be implemented entirely locally on the computer system 104. The detail enhancement model 150 may be trained at a server 106 and deployed to the computer system 104. In some embodiments, the detail enhancement model 150 is trained and applied locally at the computer system 104.

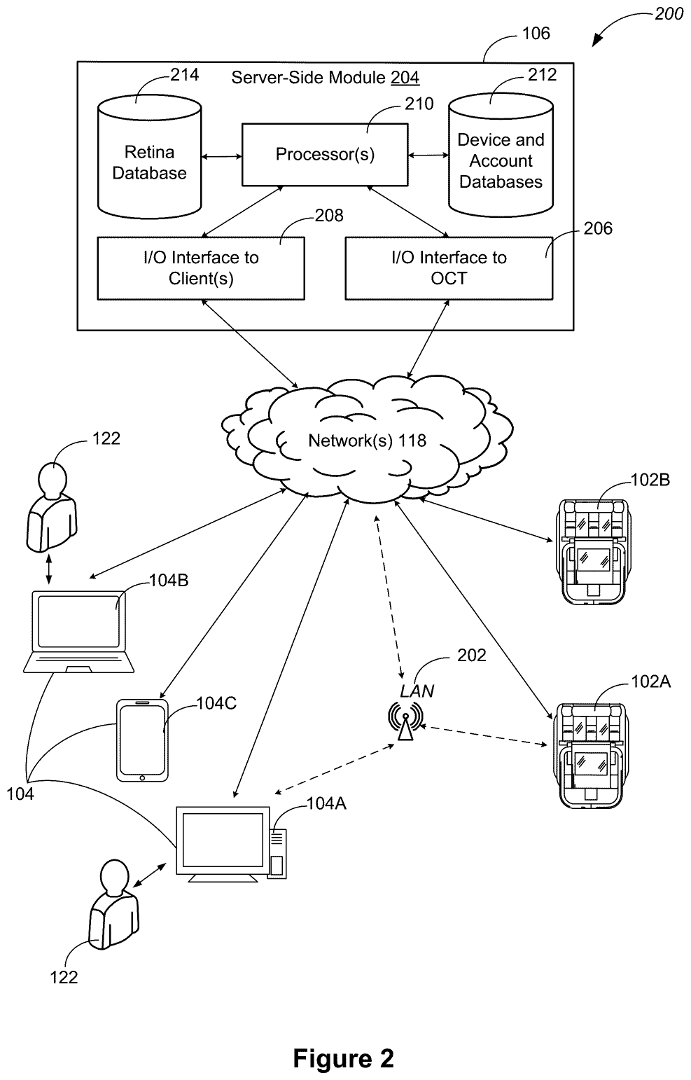

FIG. 2 is an example data processing environment 200 that facilitates communication and processing of retinal data, in accordance with some embodiments. The data processing environment 200 includes a plurality of networked OCT devices 102 (e.g., devices 102A and 102B), a plurality of networked computer devices 104 (e.g., devices 104A, 104B, and 104C), and a server 106. The OCT devices 102, computer devices 104, and server 106 are communicatively coupled to each other via one or more communication networks 118. In an example, two or more devices (e.g., an OCT device 102A and a computer device 104A) are located in close proximity to each other, such that they can be communicatively coupled in the same sub-network via wired connections or via a LAN 202 enabled by a network interface device. Each of the OCT devices 102, computer devices 104, and server 106 is configured to execute a respective eye monitoring application for scanning a retina, analyzing eye data, or reporting retinal analytic results.

The server 106 includes a server-side module 204 configured to execute a server-side eye monitoring application for generating a ROTA map 110 of an inner retinal layer from a plurality of cross-sectional scan images 108, training one or more machine learning models 120 or a detail enhancement model 150, applying the detail enhancement model 150 to enhance a level of detail of the ROTA map 110, and/or applying the machine learning models 120 to implement one or more retinal analytic tasks (e.g., determining visual field sensitivity 112 of a retina). The server-side module 204 of the server 106 includes input/output (I/O) interfaces 206 to the OCT devices 102, I/O interfaces 208 to the computer devices 104, one or more processors 210, a device and account database 212, and an eye database 214. An I/O interface to one or more OCT devices 102 facilitates input and output processing of the plurality of scan images 108 for the server-side module 204. An I/O interface to one or more computer devices 104 facilitates input and output processing of the scan images 108, ROTA images 110, machine learning models, or analytic results 112-116 for the server-side module 204. The device and account database 212 stores a plurality of profiles for reviewer or patient accounts registered with the server 106. Each user profile includes account credentials for a respective reviewer or patient account. The eye database 214 stores the scan images 108, ROTA images 110, and/or analytic results 112-116, as well as distinct types of metadata for use in data processing for eye monitoring and diagnostic evaluation for each reviewer or patient account.

Each OCT device 102 is configured to execute an eye monitoring application to capture/acquire a plurality of scan images 108 of a retina. The OCT device 102 optionally sends the plurality of scan images 108 to a local computer device 104A or a remote server 106. In some embodiments, the computer device 104A is configured to execute an eye monitoring application to obtain the plurality of cross-sectional scan images 108 of the retina and generate a ROTA map 110 of the inner retinal layer from the plurality of cross-sectional scan images 108. In some embodiments, the computer device 104B is configured to execute an eye monitoring application to obtain the ROTA map 110 and implement one or more retinal tasks using the one or more machine learning models 120 provided by the server 106. Further, In some embodiments, the computer device 104B is configured to execute an eye monitoring application to obtain the ROTA map 110 and apply a detail enhancement model 150 to enhance a level of detail of the ROTA map 110 before implementing any retinal analytic tasks. In some embodiments, a computer device 104C is configured to execute an eye monitoring application to obtain the plurality of scan images 108, ROTA map 110, visual field sensitivity 112 of the retina, probability 114 of each of one or more eye diseases, defect location 116 in the RNFL, or any other retinal analytic results, and present such retinal analytic results to a doctor 122 or to a patient for review. In some embodiments, a subset of the retinal analytic results (e.g., visual field sensitivity 112) is visualized in a graphical user interface (GUI) of the computer device 104C.

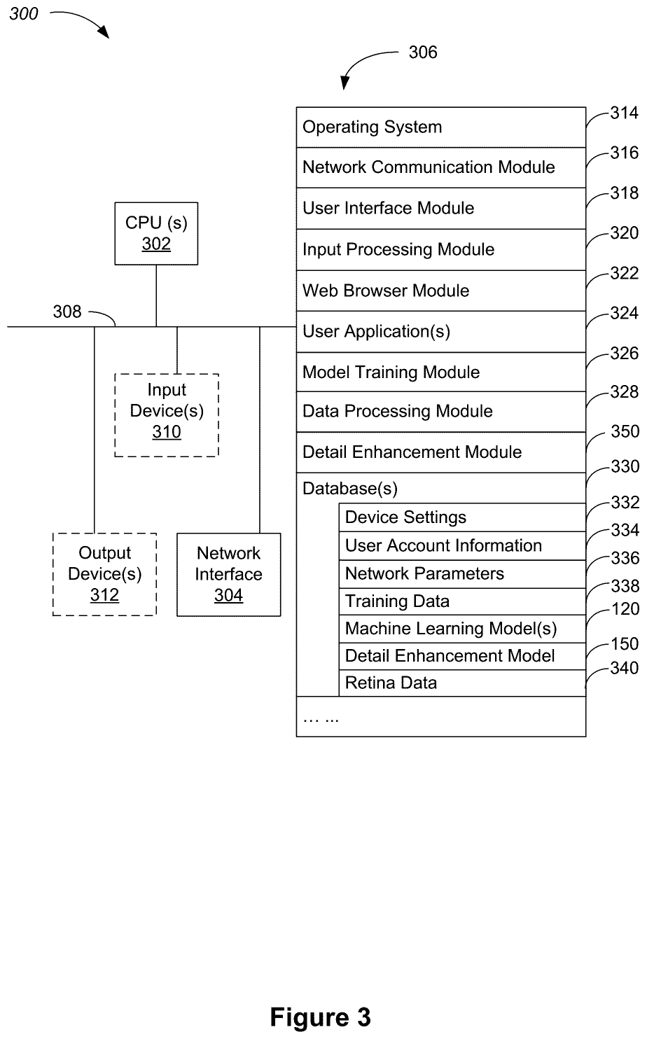

FIG. 3 is a block diagram of a computer system 300 configured to increase optical textural details of the retinal nerve fiber bundles on a ROTA map generated from a plurality of OCT scan images 108 of the retina, in accordance with some embodiments. In some embodiments, the computer system 300 includes a server 106, an OCT device 102, a first computer device 104A, a second computer device 104B, or a combination thereof. The computer system 300 typically includes one or more processing units (CPUs) 302, one or more network interfaces 304, memory 306, and one or more communication buses 308 for interconnecting these components (sometimes called a chipset). The computer system 300 includes one or more input devices 310 that facilitate user input, such as a keyboard, a mouse, a voice-command input unit or microphone, a touch screen display, a touch-sensitive input pad, a gesture capturing camera, or other input buttons or controls. Furthermore, in some embodiments, the computer system 300 uses a microphone and voice recognition or a camera and gesture recognition to supplement or replace the keyboard. In some embodiments, the computer system 300 includes one or more cameras, scanners, or photo sensor units. The computer system 300 also includes one or more output devices 312 that enable presentation of user interfaces and display content, including one or more speakers and/or one or more visual displays.

The memory 306 includes high-speed random-access memory, such as DRAM, SRAM, DDR RAM, or other random-access solid-state memory devices. In some embodiments, the memory 306 includes non-volatile memory, such as one or more magnetic disk storage devices, one or more optical disk storage devices, one or more flash memory devices, or one or more other non-volatile solid state storage devices. In some embodiments, the memory 306 includes one or more storage devices remotely located from one or more processing units 302. The memory 306, or alternatively the non-volatile memory within the memory 306, includes a non-transitory computer readable storage medium. In some embodiments, the memory 306, or the non-transitory computer readable storage medium of the memory 306, stores the following programs, modules, and data structures, or a subset or superset thereof:
an operating system 314, which includes procedures for handling various basic system services and for performing hardware dependent tasks;
a network communication module 316, which connects each OCT device 102, computer device 104, or server 106, to other devices via one or more network interfaces 304 (wired or wireless) and one or more communication networks 118, such as the Internet, other wide area networks, local area networks, metropolitan area networks, and so on;
a user interface module 318, which enables presentation of information (e.g., a graphical user interface for application(s) 324, widgets, websites and web pages thereof, and/or games, audio and/or video content, or text) at each OCT device 102, computer device 104, or server 106 via one or more output devices 312 (e.g., displays or speakers);
an input processing module 320, which detects one or more user inputs or interactions from one of the one or more input devices 310 and interprets the detected input or interaction;
a web browser module 322, which navigates, requests (e.g., via HTTP), and displays websites and web pages thereof, including a web interface for logging into a user account associated with an OCT device 102, a computer device 104, a server 106, or another electronic device. The web browser 322 may control the OCT device 102 or the computer device 104 when associated with a user account, enabling a user to edit and/or review settings and data that are associated with the user account;
one or more user applications 324, which execute by the computer system 300 (e.g., an eye monitoring application or other web or non-web-based applications for reviewing scan images 108, ROTA maps 110, or enhanced ROTA maps 124 with increased optical textural details of retinal nerve fiber bundles);
a model training module 326, which receives training data including pairs of training ROTA maps. The training module 326 builds a machine learning model 120 for processing ROTA maps 110 generated from scan images 108 of a retina;
a data processing module 328, which receives scan images 108 and/or ROTA maps 110 of a retina, where in some embodiments, the data processing module 328 is associated with an eye monitoring application that enhances a level of detail of a ROTA map 110 (e.g., using a detail enhancement model 150 (FIG. 1)) or implements a task for scanning a retina, analyzing eye data, and/or reporting retinal analytic results (e.g., using a machine learning model 120 (FIG. 1));
a detail enhancement module 350 for enhancing one or more optical textural details of a first visual representation (e.g., a ROTA map) a patient's retina, e.g., using a detail enhancement model 150 (FIG. 1);
one or more databases 330, which store one or more of:
   device settings 332, including common device settings (e.g., service tier, device model, storage capacity, processing capabilities, and/or communication capabilities) of each OCT device 102, computer device 104, or server 106;
   user account information 334 for the one or more user applications 324 (e.g., user names, security questions, account history data, user preferences, and predefined account settings);
   network parameters 336 for the one or more communication networks 118 (e.g., IP address, subnet mask, default gateway, DNS server, and host name);
   training data 338 for training one or more machine learning models 120. The training data 338 includes a plurality of pairs of training ROTA maps;
   machine learning models 120 for processing ROTA maps 110 with or without enhancement and implementing one or more retinal analytic tasks (e.g., determining visual field sensitivity 112 of a retina);
   detail enhancement model 150 for processing ROTA maps 110 determined from scan images 108 of a retina and enhancing levels of detail of the ROTA maps 110; and
   eye data 340, including at least cross-sectional scan images 108 of a retina acquired by an OCT device 102, ROTA maps 110 of an inner retinal layer, and retinal analytic results of one or more retinal analytic tasks.

Each of the above identified elements may be stored in one or more of the previously mentioned memory devices, and corresponds to a set of instructions for performing a function described above. The above identified modules or programs (i.e., sets of instructions) need not be implemented as separate software programs, procedures, modules or data structures, and thus various subsets of these modules may be combined or otherwise re-arranged in various embodiments. In some embodiments, the memory 306 stores a subset of the modules and data structures identified above. In some embodiments, the memory 306 stores additional modules and data structures not described above.

An example algorithm of optical texture analysis is described in U.S. application Ser. No. 16/159,476, “Optical Texture Analysis of Inner Retina,” which is incorporated by reference in its entity. An optical texture analysis map is a topographic display of a set of optical texture signatures derived from optical texture analysis of a retinal tissue layer in the inner retina of an eye. According to the algorithm of optical texture analysis, the inner retina is scanned by an optical coherence tomography (OCT) imaging device to obtain a plurality of 2D tomographic cross-sectional images, which forms a 3D dataset of optical reflectance measurements. The inner retina is then segmented into multiple retinal tissue layers including the retinal nerve fiber layer (RNFL), the ganglion cell layer (GCL), and the inner plexiform layer (IPL), each with an anterior boundary and a posterior boundary. An optical texture signature value can be computed for each topographic location on one or more selected retinal tissue layers by integrating (1) OCT reflectance of the tissues between the layer boundaries and (2) the tissue thickness data of the layer using a specific set of non-linear transformation and normalization processing. The optical texture signature values computed from all scanned topographic locations are then combined to derive an optical texture analysis map image for the one or more selected retinal tissue layers. ROTA is an implementation of optical texture analysis of the inner retina on the RNFL.

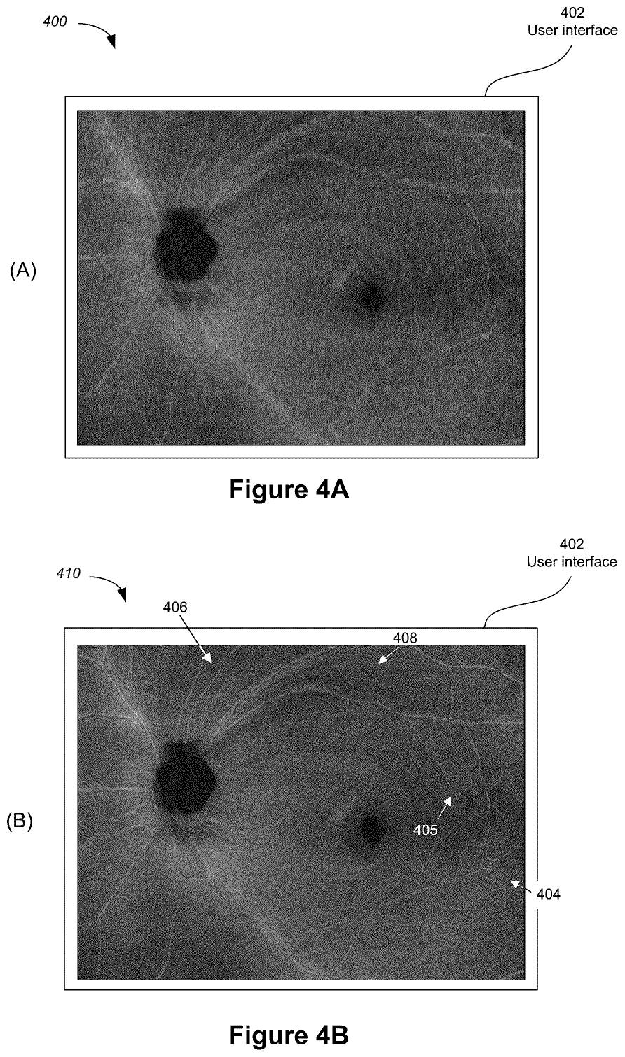

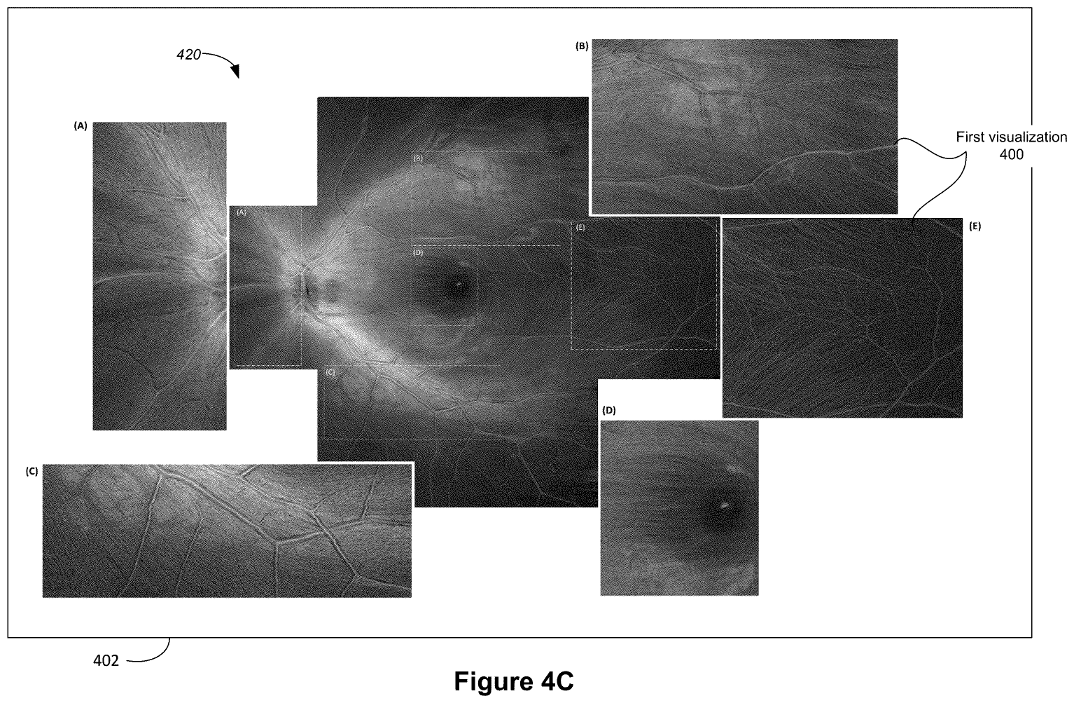

FIGS. 4A to 4C are example user interfaces showing visual representations including optical textural details, in accordance with some embodiments. Depending on the specification of an OCT imaging device or scanning protocol, ROTA maps derived from the OCT scans can show distinct levels of optical textural details of retinal axonal fiber bundles. More specifically, FIG. 4A shows a user interface 402 displaying a first visual representation 400 (e.g., a ROTA map of an eye with low optical textural details of retinal axonal fiber bundles), and FIG. 4B shows the user interface 402 displaying a second visual representation 410 (e.g., a ROTA map with high optical textural details of retinal axonal fiber bundles from the same scanned region of the eye). In other words, in some embodiments, both the first visual representation 400 and the second visual representation 410 may be provided by the same OCT imaging device 102 or two different OCT imaging devices 102 directly. When a retinal region is scanned with different scan resolutions, the resulting ROTA maps 110 would result in distinct levels of optical textural details of retinal axonal fiber bundles. For instance, a lower OCT scan resolution with 128 B-scans, each consisting of 512 A-scans, for a 12 mm×9 mm retinal region results in a ROTA map as shown in FIG. 4A with a relatively low optical textural details, while a higher scan resolution with 256 B-scans, each consisting of 512 A-scans, for the same retinal region results in a ROTA map as shown in FIG. 4B with higher optical textural details of axonal fiber bundles.

In some embodiments, an OCT imaging device 102 provides the first visual representation 400 of retinal nerve fiber bundles of a retina. The first visual representation 400 indicating optical textural details of the retinal nerve fiber bundles of the retina with a first level of detail. The first visual representation 400 is processed by a detail enhancement model 150 to generate the second visual representation 410 of the retinal nerve fiber bundles of the retina. In some embodiments, after the second visual representation 410 is generated from the first visual representation 400, one or both of the visual representations 400 and 410 are presented at the user interface 402 (or different user interfaces presented by a display of a computing device 104).

The second visual representation 410 has a higher level of detail (e.g., a higher image resolution) than the first visual representation 400. The second visual representation 410 includes a plurality of features (e.g., features 404, 405, 406, and 408), which are not detectable on the first visual representation 410. The computer system 300 may be configured to identify the plurality of features and mark the plurality of features on the second visual representation 510 explicitly (e.g., using arrows) for a reviewer. The second visual representation 410 may be applied to determine a health condition associated with the retina, e.g., based on the plurality of features.

FIG. 4C shows the user interface 402 displaying a plurality of first visual representations 400 corresponding to OCT scans of different retinal regions, in some embodiments. In some embodiments, an OCT imaging device 102 provides the plurality of first visual representations 400 of retinal nerve fiber bundles of a retina, and the plurality of first visual representation 400 correspond to different retinal regions. Each first visual representations 400 has a first level of detail. In some embodiments, the plurality of first visual representations 400 are combined to generate a first composite visual representation 420 having the first level of detail. The first composite visual representation 420 is processed with a detail enhancement model 150 to generate a second composite visual representation (not shown) having enhanced optical textural details of different retinal regions. The second composite visual representation is used to determine a retina-related health condition.

Alternatively, in some embodiments, each first visual representations 400 is processed with a detail enhancement model 150 separately to generate a respective second visual representation 410 having enhanced optical textural details of a respective retinal region. The respective second visual representation 410 has a second level of detail higher than the first level of detail. Respective second visual representation 410 generated from the plurality of first visual representation 400 are combined to generate a second composite visual representation (not shown) for use to determine a retina-related health condition.

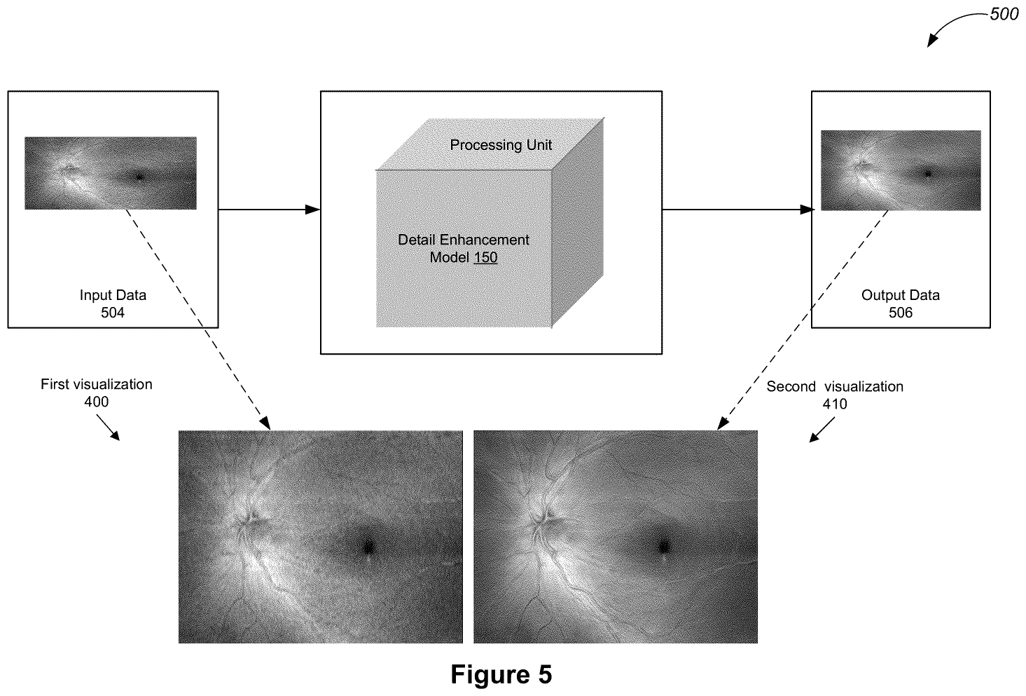

FIG. 5 depicts an example computer-implemented system 500 for enhancing optical textural details of retinal features using a detail enhancement model 150, in accordance with some embodiments. Embodiments of the present invention include a computer-implemented program as depicted in FIG. 5. The computer-implemented program comprises an input unit 504 which includes at least the first visual representation 400 (e.g., a portion of a ROTA map), a processing unit configured to apply the detail enhancement model 150, and an output unit 506 which includes the second visual representation 410 (e.g., an enhanced ROTA map with increased optical textural details of axonal fiber bundles) that is generated by the detail enhancement model 150. In some embodiments, the detail enhancement model 150 includes a trained deep neural network with at least an input layer connecting to the input unit of the computer-implemented program and an output layer connecting to the output unit of the computer-implemented program. The deep neural network may also include a set of intermediate layers having a set of weights trainable by a model training module 326 (FIG. 3).

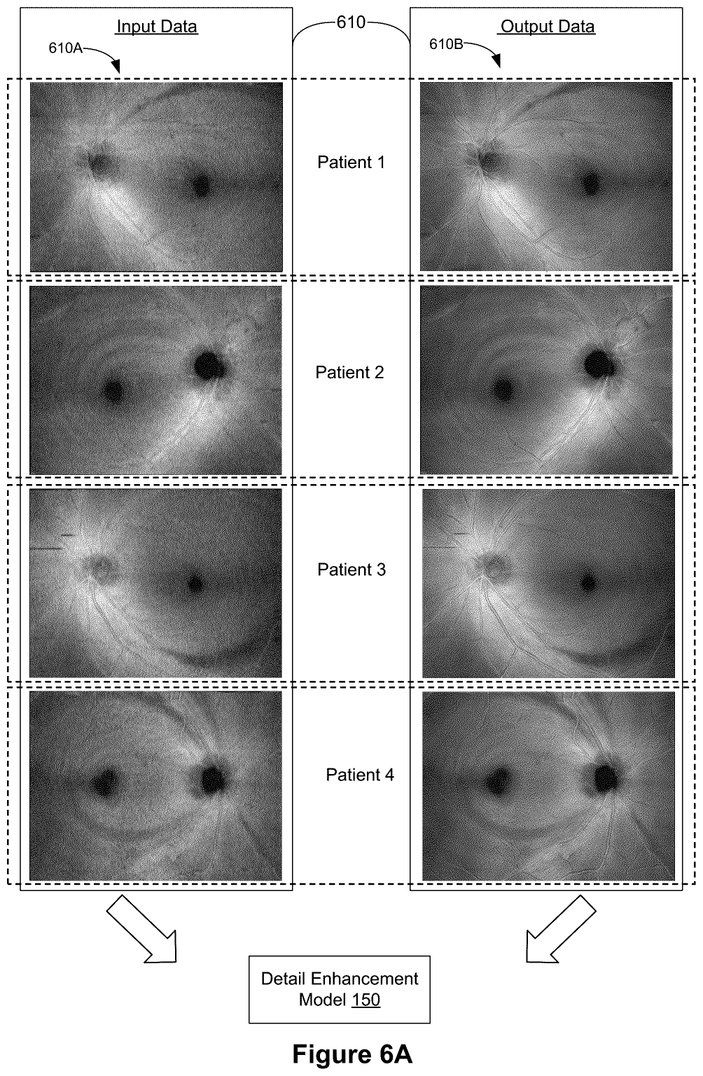

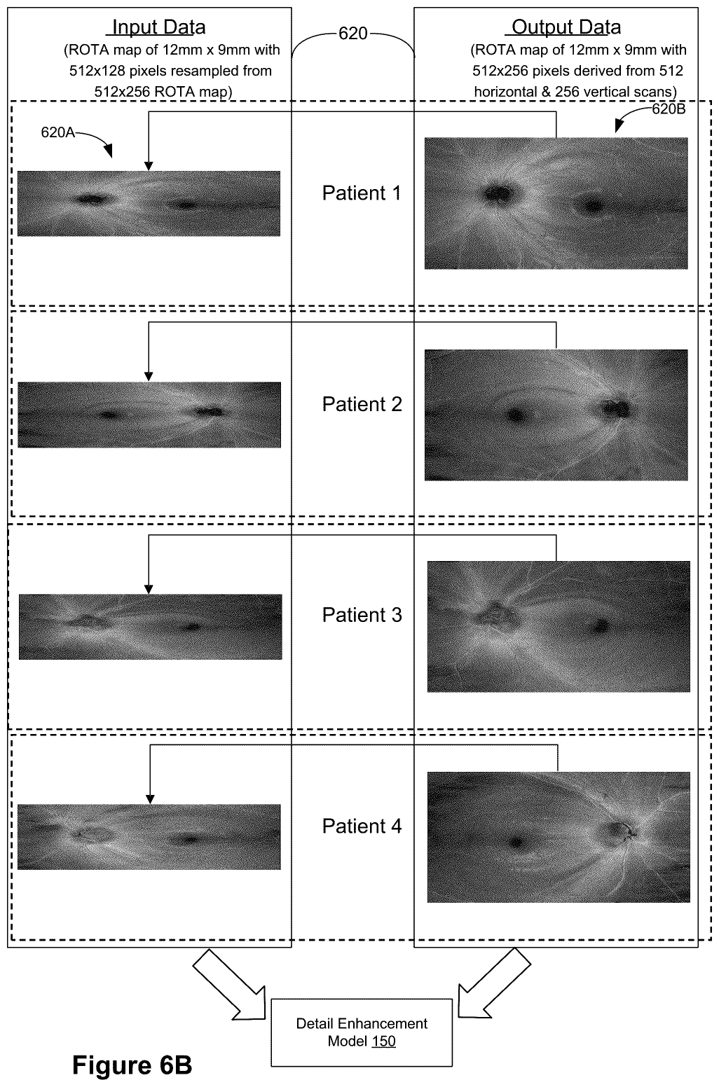

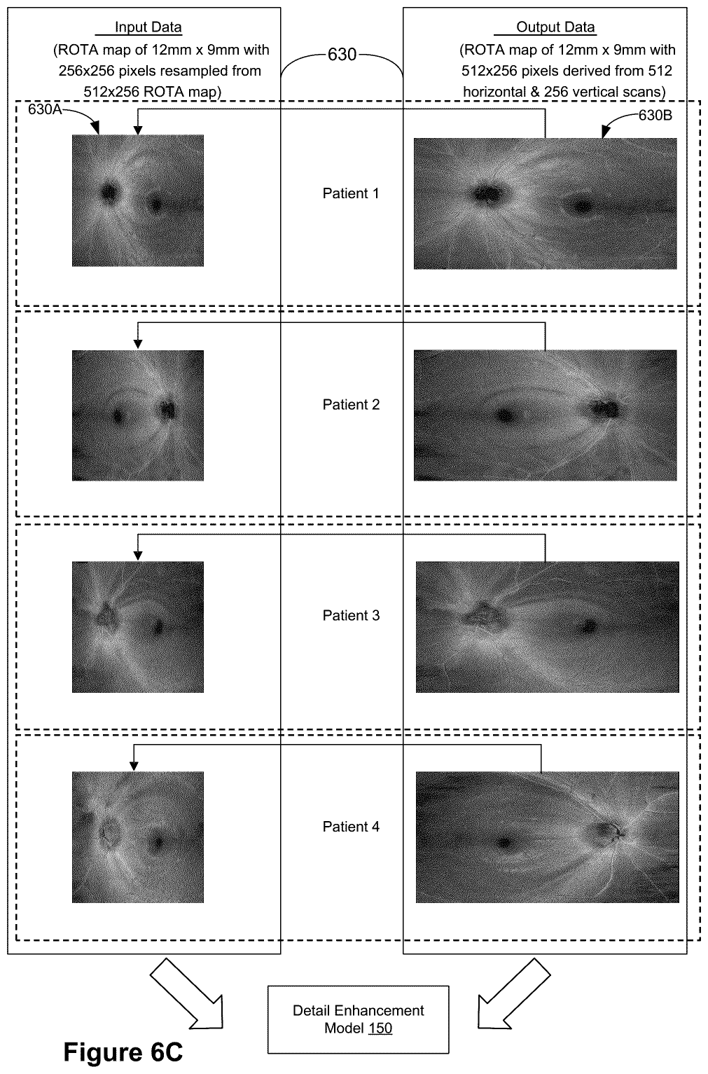

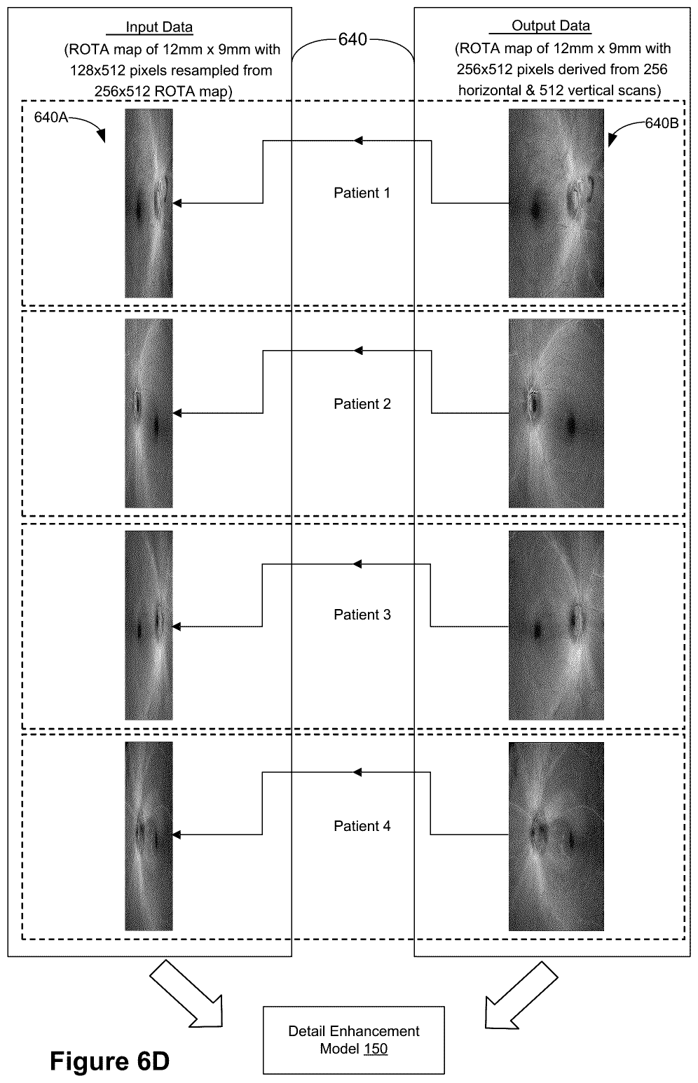

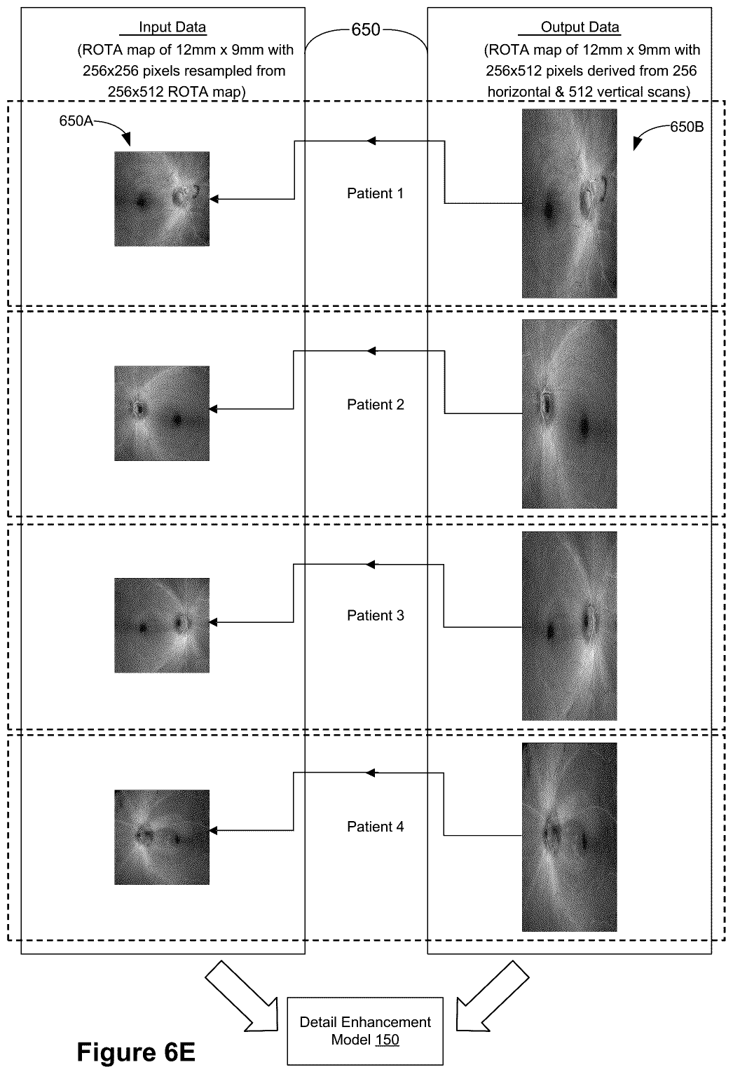

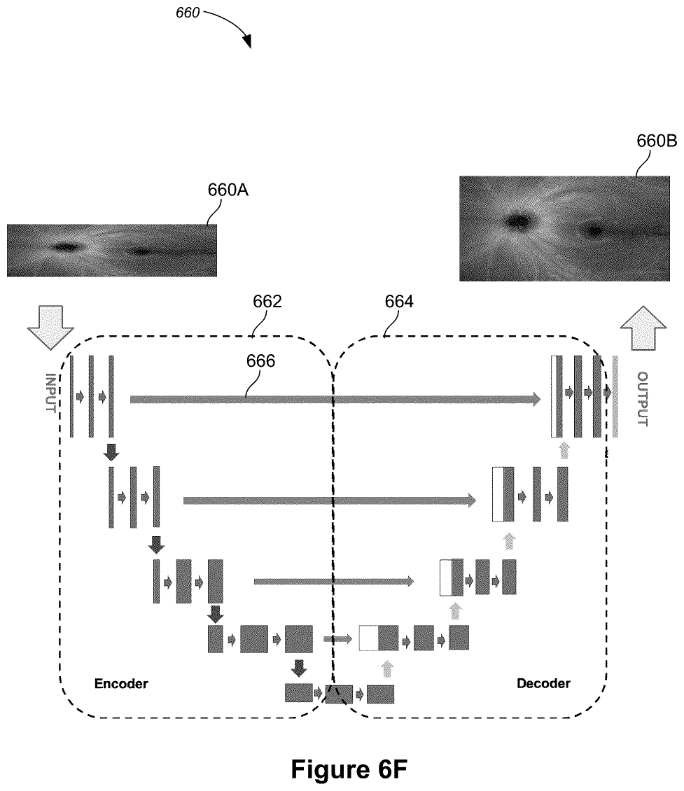

FIGS. 6A to 6E illustrate example training data 610, 620, 630, 640, and 650 used to train a detail enhancement model 150 for enhancing optical textural details of retinal features, in accordance with some embodiments. For example, FIG. 6A illustrates a plurality of paired data samples 610. In some embodiments, the trainable weights can be trained in a supervised manner, using the plurality of paired data samples. A set of ROTA maps 610A have low optical textural details (or inferior visualization) of retinal axonal fiber bundles, and are used as model input data. Each ROTA map 610A having the low optical textural details corresponds to a respective ROTA map 610B of the same scanned region with high optical textural details (or superior visualization) of retinal axonal fiber bundles, and the respective ROTA map 610B is applied as output data of the detail enhancement model 150. Stated another way, the respective ROTA map 610B is used as ground truth.

In some embodiments, the intermediate layers of the deep neural network include, but are not limited to, one or more of the following: a convolutional neural network including one or more convolutional blocks; a transformer neural network including one or more attention blocks; and a multi-layered perceptron neural network including one or more multi-layer perceptron blocks.

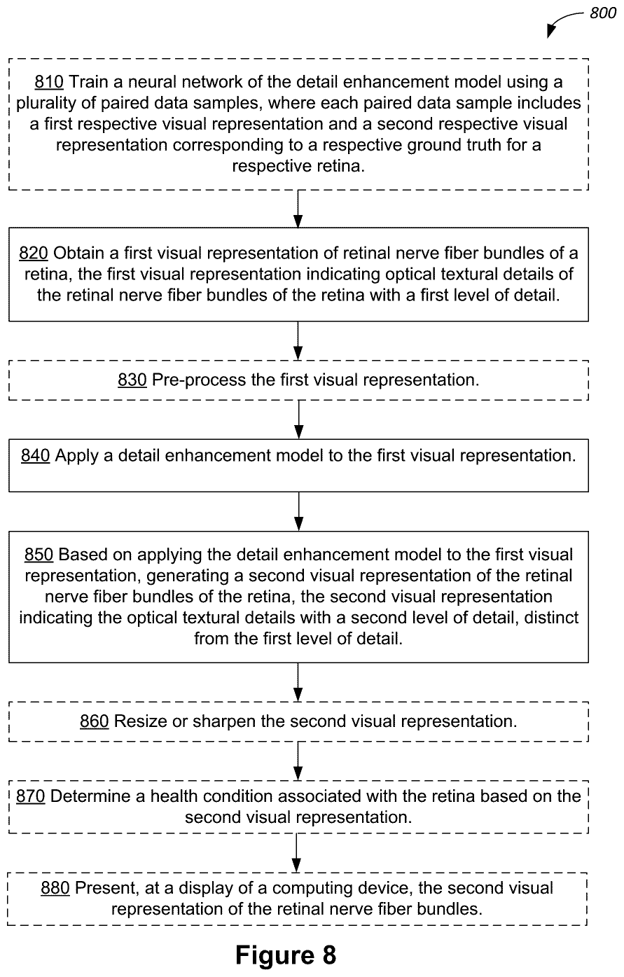

With the trained machine learning model, the workflow as shown in FIG. 8 can be implemented for prediction on new visual representations (e.g., ROTA maps) to increase their optical textural details. After a new OCT scan set is captured from an eye and a new ROTA map is derived from the OCT scan set, the ROTA map image is received by the trained machine learning model at the input unit. The detail enhancement model 150 then processes the ROTA map to predict the higher optical textural details at each scanned location and generate an enhanced version of the ROTA map at the output unit with increased optical texture details of retinal axonal fiber bundles. In some embodiments, the enhanced ROTA map generated at the output unit has a higher image resolution than the original ROTA map at the input unit.

The training dataset can be composed by various methods. In some embodiments, the training input sample (e.g., ROTA maps 610A with low optical textural details of axonal fiber bundles as model input in a training data pair) is an image resampled with lower image resolution from the training output sample (e.g., ROTA maps 610B with high optical textural details of axonal fiber bundles as model output in a training data pair). Stated another way, in some embodiments, the ROTA maps 610B are provided by an alternative OCT imaging device configured to obtain scans with high optical textural details of axonal fiber bundles. The ROTA maps 610B are down-sampled to generate the ROTA maps 610A having a lower level of detail of a target OCT imaging device. The ROTA maps 610A and 610B are applied to train the detail enhancement model 150 applicable to process ROTA maps 110 derived from OCT scans of the target OCT imaging device.

In some embodiments, the ROTA maps 610A are generated by down-sampling the ROTA maps 610B generated based on OCT scans, and have lower resolutions than the ROTA maps 610B. Alternatively, in some embodiments, the ROTA maps 610A have the same resolutions as the ROTA maps 610B. The ROTA maps 610B having a higher level of detail may be processed to decrease visibility of some features (e.g., features 404-408 in FIG. 4) to generate the ROTA maps 610A. In some situations, noise is added to the ROTA maps 610B having the higher level of detail to generate the ROTA maps 610A. During training, the ROTA maps 610A are further applied as inputs to the detail enhancement model 150, and the ROTA maps 610B are applied as ground truth. The detail enhancement model 150 is applied to process the ROTA maps 610A iteratively to generate enhanced ROTA maps, which are compared to the ROTA maps 610B. Weights of the detail enhancement model 150 are adjusted to control a difference (also called a loss) between the enhanced ROTA maps and the ROTA maps 610B.

FIG. 6B illustrates a set of example pairs 620 of input ROTA map 620A and output ROTA map 620B from the training dataset to be used in deep learning to train the detail enhancement model 150, in accordance with some embodiments. Each output ROTA map 620B in the training dataset has an image resolution of 512×256 pixels and is derived from an OCT scan set for a 12 mm×9 mm region with scan resolution of 256 horizontal B-scans with each consisting of 512 A-scans captured from a patient's eye. The corresponding paired input ROTA map 620A is then obtained by resampling 512×128 pixels from the output ROTA map 620B, which synthesizes a ROTA map with relatively low optical textural details of axonal fiber bundles for the same 12 mm×9 mm region.

FIG. 6C illustrates paired ROTA maps 630A and 630B. The input ROTA map 630A is obtained by resampling 256×256 pixels from the output 512×256 ROTA map 630B.

FIG. 6D illustrates paired ROTA map 640A and 640B. Each output ROTA map 640B in the training dataset has an image resolution of 256×512 pixels and is derived from an OCT scan set for a 12 mm×9 mm region with scan resolution of 256 vertical B-scans with each consisting of 512 A-scans captured from a patient's eye. The corresponding paired input ROTA map 640A is obtained by resampling 128×512 pixels from the output ROTA map sample 640B.

FIG. 6E illustrates paired input ROTA maps 650A and 650B. Each input ROTA map 650A can be obtained by resampling 256×256 pixels from a respective output 256×512 ROTA map 650B. In other embodiments, the training dataset can be composed by using a similar resampling approach on ROTA maps derived from OCT scans captured by a different model/variety of OCT imaging device with a different scan resolution or a distinct size of scanned region. In other words, the scan resolution, and the size of scanned region of the input and output ROTA maps in the training dataset are subject to the specifications of the OCT imaging devices and their scan protocols.

In some embodiments, any of the input ROTA maps 610A to 650A can be optionally processed at the input unit before received by the machine learning model during both training and prediction, and the processing may include, but not limited to, any of the following: resizing to an image resolution same as model output with nearest neighbor interpolation; resizing to an image resolution same as model output with bilinear interpolation; resizing to an image resolution same as model output with bicubic or cubic spline interpolation; interlacing with alternating blank lines between each pair of neighbor rows of pixels; interlacing with alternating blank lines between each pair of neighbor columns of pixels; noisifying; blurring; cutout of pixels at various locations; or a combination of any of the above.

FIG. 6F depicts an example process 660 for enhancing optical textural details of retinal features in an input ROTA map using a U-Net, in accordance with some embodiments. A detail enhancement model 150 includes the U-Net, and is applied to provide an output ROTA map 660B including higher optical textural details based on an input ROTA map 660A. In some embodiments, the detail enhancement model 150 generates feature patterns in ROTA maps 660B having higher optical textural details based on feature patterns in ROTA maps 660A having lower optical textural details. The detail enhancement model 150 is trained to generate the enhanced ROTA map 660B at its final layer. In some embodiments, a machine learning model corresponding to the detail enhancement model 150 is constructed by, but not limited to, one of the follows: (1) a deep neural network with architecture based on U-Net, comprising at least one encoder 662 and one decoder 664 with skip-connections 666 or cross-connections from one or more layers in the encoder 662 to one or more layers in the decoder 664; (2) an ensemble network formed by more than one U-Net based neural networks; (3) a deep neural network (not shown) with architecture based on Generative Adversarial Network (GAN), comprising at least one generator sub-model and one discriminator sub-model; (4) an ensemble network formed by more than one GAN-based neural networks; and (5) an ensemble network formed by a combination of any two of the above.

The enhanced ROTA map 660B can be optionally processed at the output unit after being generated by the detail enhancement model 150. In some embodiments, the processing may include, but not limited to, image resizing, image sharpening by unsharp masking, or a combination of these processing. In some embodiments, the enhanced ROTA map 660B can be passed into the input unit and processed by the one or more machine learning models again for further enhancement at least once. In one of the embodiments, the enhanced ROTA map 660B can be directly passed into the input unit again. In another one of the embodiments, the enhanced ROTA map 660B can be resampled to an image with lower resolution before passing into the input unit again.

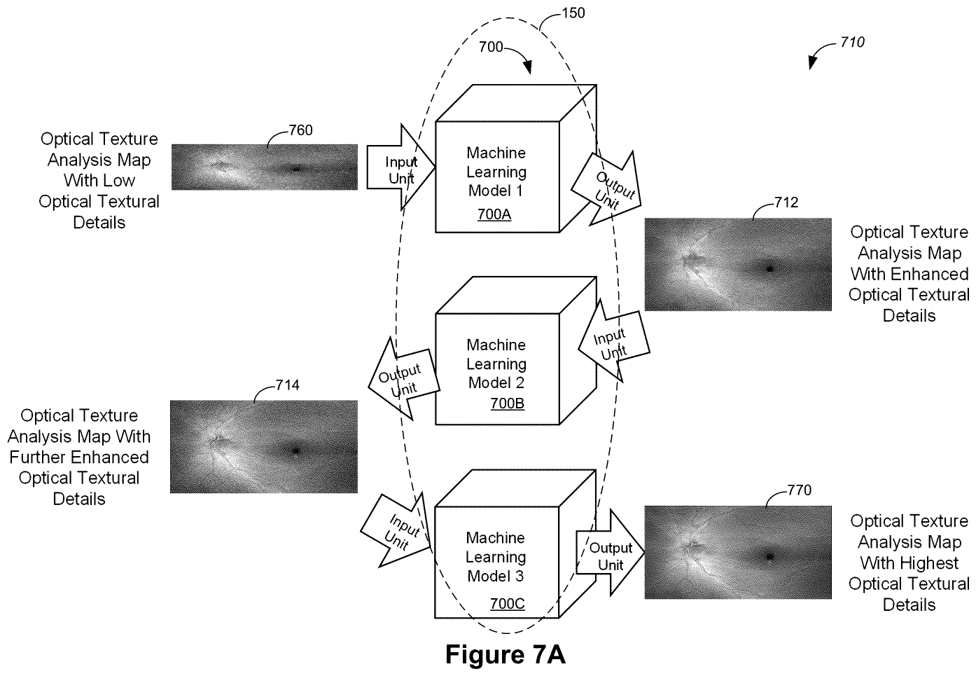

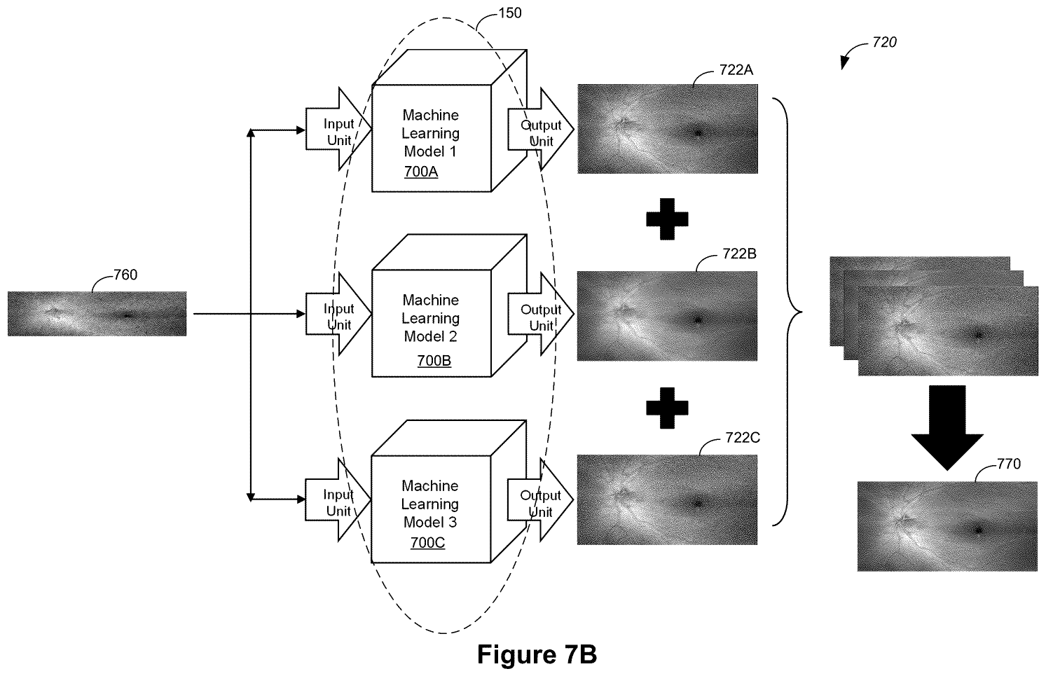

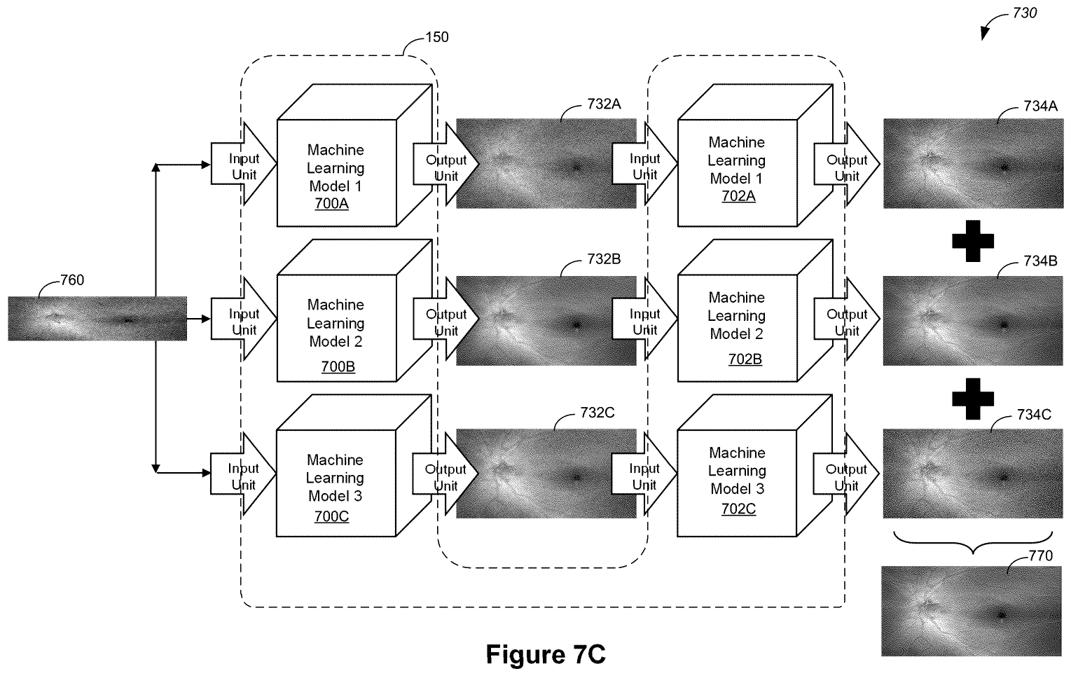

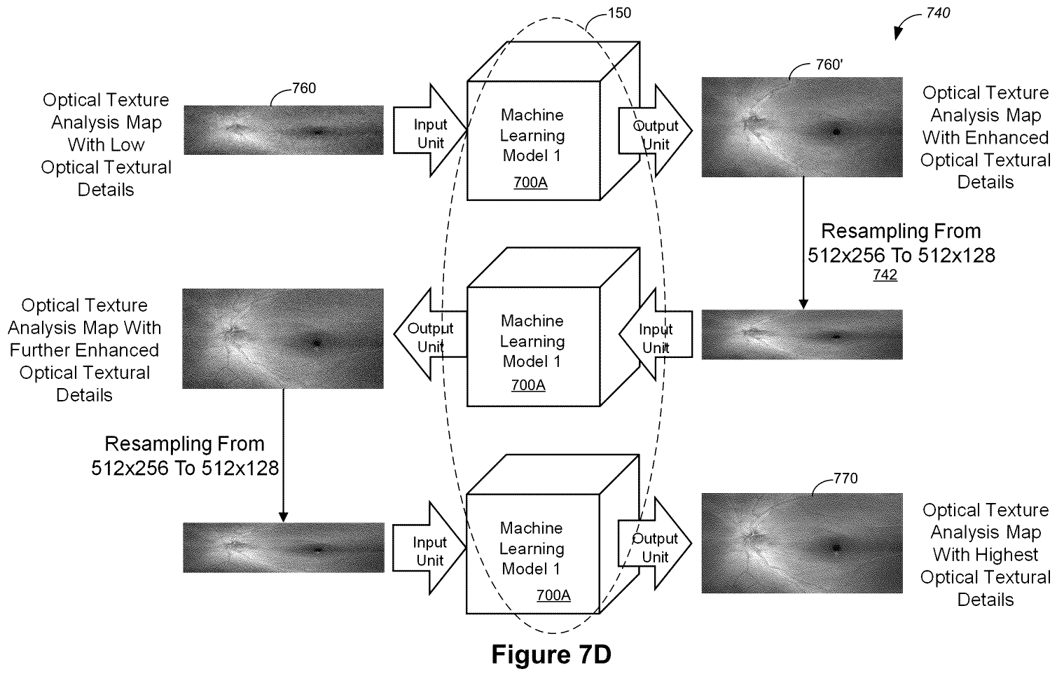

FIGS. 7A to 7D are example processes 710, 720, 730, and 740 of enhancing a detail level of a first visual representation 760 (e.g., representation 400 in FIG. 4A) of a retina with a detail enhancement model 150 including a plurality of machine learning models 700 (e.g., 700A, 700B, 700C), in accordance with some embodiments. A second visual representation 770 (e.g., representation 410 in FIG. 4B) is generated with a higher level of detail (e.g., a higher image resolution, or having additional features). Referring to FIG. 7A, in some embodiments, the first visual representation 760 is successively processed by a plurality of distinct machine learning models 700A, 700B, and 700C to generate the second visual representation 770. In some embodiments, the machine learning models 700A-700C are identical, and enhance a first level of detail of the first visual representation 760 without changing its image resolution (e.g., by adding additional details, reducing noise, and correcting image artifacts).

Referring to FIG. 7B, in some embodiments, the first visual representation 760 is processed by a plurality of distinct machine learning models 700A, 700B, and 700C in parallel to generate three intermediate visual representations 722A, 722B, and 722C, which are combined to generate the second visual representation 770. Referring to FIG. 7C, in some embodiments, the first visual representation 760 is processed by a plurality of distinct machine learning models 700A, 700B, and 700C in parallel to generate three intermediate visual representations 732A, 732B, and 732C, which are further processed by the plurality of distinct machine learning models 702A, 702B, and 702C in parallel to generate three additional visual representations 734A, 734B, and 734C. The three additional visual representations 734A, 734B, and 734C are combined to generate the second visual representation 770. In some embodiments, one or more of machine learning models 702A-702C is the same as one or more of machine learning model 700A to 700C. Referring to FIG. 7D, in some embodiments, the first visual representation 760 is successively processed by a machine learning model 700A to generate the second visual representation 770. In some embodiments, one or more of machine-learning models 730A to 730C is the same as one or more of the machine learning models 700A to 700C. After the first visual representation 760 is processed by the machine learning model 700A, the processed first visual representation 760′ is down-sampled (operation 742) and applied as an input to the machine learning model 700A.

In some embodiments, more than one machine learning models 700 can be implemented at the processing unit of the computer-implemented program. Multiple machine learning models 700 which can individually increase the optical textural details of retinal axonal fiber bundles on ROTA maps 110 are trained. In some embodiments, the enhanced ROTA map (e.g., visual representations 732A-732C in FIG. 7B) generated by one machine learning model can be received by the other machine learning models one-by-one for serial enhancement. In some embodiments (FIG. 7B), the multiple machine learning models 700 can process the same input ROTA map (e.g., first visual representation 760) for parallel enhancement and generate multiple enhanced ROTA maps (e.g., second visual representation 770). The multiple enhanced ROTA maps are then overlaid so that each pixel location has multiple pixel values readable from the multiple enhanced ROTA maps. In an example, the multiple pixel values at each pixel location then averaged to one final value for that pixel location and an averaged ROTA map can be composed. In another example, different weights are assigned to the multiple enhanced ROTA maps such that a weighted average value is computed from the multiple pixel values at each pixel location, and a weighted-average ROTA map can be composed.

The machine learning model trained by ROTA maps derived from OCT scans captured by one model/variety of OCT imaging device can be applied on new ROTA maps derived from the same model/variety of OCT imaging device. In some embodiments, the trained machine learning model can be applied on a new ROTA map with low optical textural details of axonal fiber bundles derived from OCT scans captured by the same model/variety of OCT imaging device for increasing the optical textural details. In some embodiments (FIG. 7A), a trained machine learning model (e.g., model 700B or 700C) can be further applied on a new ROTA map (e.g., representation 712 or 714) with high optical textural details of axonal fiber bundles derived from OCT scans captured by the same model/variety of OCT imaging device for further enhancing the optical textural details.

Alternatively, the machine learning model trained by ROTA maps derived from OCT scans captured by one model/variety of OCT imaging device is not limited to be applied on new ROTA maps derived from the same model/variety of OCT imaging device, but can be generalized to ROTA maps from a different model/variety of OCT imaging device. In some embodiments, the trained machine learning model can be applied on a new ROTA map with low optical textural details of axonal fiber bundles derived from OCT scans captured by a different model/variety of OCT imaging device for increasing optical textural details. In some embodiments, the trained machine learning model can be applied on a new ROTA map with high optical textural details of axonal fiber bundles derived from OCT scans captured by a different model and/or a variety of OCT imaging device for further enhancing the optical textural details.

In some embodiments, the machine learning model 700 is trained by ROTA maps derived from OCT scans captured by more than one models/varieties of OCT imaging devices to further enhance the generalization capability.

The machine learning model 700 can be deployed for processing by various means. In some embodiments, the machine learning model 700 is deployed on a local computer 104 or server 106 (e.g., in the same geometrical location) as the eye being examined and scanned. In some embodiments, the machine learning model 700 is deployed on a cloud computing platform where the input ROTA map 110 is uploaded through the Internet.

In one or more examples, the functions described may be implemented in hardware, software, firmware, or any combination thereof. When implemented in software, the functions may be stored on or transmitted over, as one or more instructions or code, on a computer-readable medium, and executed by a hardware-based processing unit. Computer-readable media may include computer-readable storage media, which corresponds to a tangible medium such as a data storage medium, or communication media including any medium that facilitates transfer of a computer program from one place to another (e.g., according to a communication protocol). In this manner, computer-readable media may correspond to (1) tangible computer-readable storage media, which is non-transitory or (2) a communication medium such as a signal or carrier wave. Data storage media may be any available media that can be accessed by one or more computers or one or more processors to retrieve instructions, code and/or data structures for implementation of the embodiments described in the present application. A computer program product may include a computer-readable medium.

### Example Embodiments

FIG. 8 is a flowchart illustrating a method 800 for enhancing optical textural details of retinal features in accordance with some embodiments. For convenience, the method 800 is described as being implemented by the computer system 300. In some embodiments, the method 800 is governed by instructions that are stored on a non-transitory computer-readable storage medium. The instructions are executed by one or more processors of the electronic system. In some embodiments, the method 800 is more specifically for increasing the optical textural details of the retinal axonal fiber bundles on a retinal nerve fiber layer (RNFL) optical texture analysis (ROTA) map acquired from a patient's eye by applying one or more machine learning models.

Each of the operations shown in FIG. 8 may correspond to instructions stored in computer memory or on a non-transitory computer readable storage medium (e.g., the memory 306 of the computer system 300 in FIG. 3). The computer readable storage medium may include a magnetic or optical disk storage device, solid state storage devices such as Flash memory, or other non-volatile memory device or devices. The instructions stored on the computer readable storage medium may include one or more of: source code, assembly language code, object code, or other instruction format that is interpreted by one or more processors. Some operations in the method 800 may be combined and/or the order of some operations may be changed.

In some embodiments, the computer system trains (810) a neural network of the detail enhancement model (e.g., model 150 in FIG. 1) using a plurality of paired data samples (e.g., training data 610, 620, 630, 640, and 650 in FIGS. 6A-6E), where each paired data sample includes a first respective visual representation (e.g., maps 610A, 620A, 630A, 640A, and 650A) and a second respective visual representation (e.g., maps 610B, 620B, 630B, 640B, and 650B) corresponding to a respective ground truth for a respective retina.

In some embodiments, a first respective data sample of each paired data sample includes the first respective visual representation of respective retinal nerve fiber bundles, the first respective visual representation having an input level of detail, and a second respective data sample of each of the paired data samples includes the second respective visual representation of the respective retinal nerve fiber bundles, the second respective visual representation having an output level of detail different than the input level of detail.

In some embodiments, training the neural network of the detail enhancement model further includes obtaining the second respective visual representation of each of the paired data samples and down-sampling the second respective visual representation to generate the first visual representation, where (i) the first respective data sample includes one or more down-sampled images of the second respective data sample, and (ii) the one or more down-sampled images have a lower level of detail (e.g., a lower resolution) than the second respective visual representation of the second respective data sample.

In some embodiments, a respective model of the detail enhancement model is trained by visual representations (e.g., ROTA maps 110 in FIG. 1) derived from OCT scans captured by more than one models/varieties of OCT imaging devices 102 to further enhance the generalization capability. In some embodiments, the training input sample (e.g., a ROTA map with the lower optical textural details of the axonal fiber bundles in a training data pair) is an image resampled with lower resolution from the training output sample (e.g., a ROTA map with the higher optical textural details of the axonal fiber bundles as model output in a training data pair). In some embodiments, the scan resolution, and the size of scanned region of the input and output ROTA maps in the training dataset are not limited to one particular OCT imaging device and are subject to the specifications of the OCT imaging devices and their scan protocols.

The computer system 300 obtains (820) a first visual representation (e.g., a ROTA map 110 determined based on OCT scans) of retinal nerve fiber bundles of a retina, the first visual representation indicating optical textural details of the retinal nerve fiber bundles of the retina with a first level of detail (e.g., a first resolution, a first level of sharpness). In some embodiments, the retinal fiber bundles of the retina include axonal fiber bundles of a retinal nerve fiber layer (RNFL) of the retina.

In some embodiments, the first visual representation (e.g., representations 760 in FIGS. 7A-7D) is obtained using an OCT device, such as the OCT device 102. In some embodiments, the first visual representation is a ROTA map, such as the ROTA map 110. For example, in some embodiments, the computer system 300 obtains one or more cross-sectional scan images of the retina captured by the OCT device 102, and generates, using the one or more cross-sectional scan images of the retina, the first visual representation.

In some embodiments, the ROTA map includes a plurality of pixels, and each pixel of the ROTA map includes a respective signature value S providing information about tissue composition and optical density of the inner retinal layer at a respective retinal location. In some embodiments, obtaining the first visual representation includes identifying boundaries of a retinal nerve fiber layer (RNFL) based on one or more particular threshold transitions of optical density of the retinal nerve fiber bundles in the one or more cross-sectional scan images.

In some embodiments, the computer system 300 pre-processes (830) the first visual representation, e.g., before feeding the first visual representation to a detail enhancement model 150. In some embodiments, pre-processing the first visual representation (e.g., the ROTA map) includes one or more of: (i) resizing to an image resolution that is more compatible with the detail enhancement model using one or more of nearest neighbor interpolation, bilinear interpolation, bicubic spline interpolation, and cubic spline interpolation; (ii) interlacing alternating blank horizontal lines between each vertical pair of neighbor rows of pixels of the ROTA map; and/or (iii) interlacing alternating blank horizontal lines between each vertical pair of neighbor rows of pixels of the ROTA map.

In some embodiments, the same and/or different pre-processing steps can be applied to visual representations provided as inputs during the process of training the detail enhancement model. For example, additional pre-processing of training data could include one or more of: (i) noisifying one or more respective pixels of the pixels of the ROTA map; (ii) blurring respective pixels of the ROTA map; and (iii) removing one or more respective pixels of the pixels of the ROTA map at each of one or more target locations. For example, target locations may be randomly selected.

The computer system 300 applies (840) a detail enhancement model (e.g., the detail enhancement model 150 show in FIG. 5) to the first visual representation. For example, as shown in FIG. 5, the first visual representation 400 is provided to the detail enhancement model 150 as part of the input data 504.

In some embodiments, the detail enhancement model includes a first machine learning model and a second machine-learning model, and the method further includes (i) training the first machine learning model using a first down-sampled set of images using a first sampling factor in first direction (e.g., horizontal direction), and (ii) training the second machine learning model using a second down-sampled set of images using a second sampling factor in a second direction. In some embodiments, the first sampling factor is equal to the second sampling factor. In some embodiments, the first sampling factor is different from the second sampling factor. (e.g., a vertical direction). In some embodiments, the first and second directions are substantially perpendicular (e.g., within 5, 10, 15 degrees of perpendicular).

In some embodiments, the detail enhancement model includes a neural network. In some embodiments, the architecture of the neural network is based on one of (i) U-Net and includes an encoder and a decoder, and one or more layers of the encoder is coupled to one or more layers of the decoder via at least one skip connection or at least one cross connection, and/or (ii) GAN and comprises a generator sub-model and a discriminator sub-model.

In some embodiments, the detail enhancement model includes a trained deep neural network, that includes (i) an input layer at the first layer of the neural network which receives a ROTA map image as model input; (ii) an output layer at the final layer of the neural network which generates an enhanced ROTA map image with increased optical textural details of axonal fiber bundles as model output; and (iii) a set of intermediate layers, with a set of trainable weights in the intermediate layers trained with deep learning method using a training dataset comprised from a plurality of paired data samples, in which a ROTA map with lower optical textural details of the axonal fiber bundles as model input is paired with a ROTA map of the same scanned region with higher optical textural details of the axonal fiber bundles as model output. In some embodiments, the intermediate layers of the machine learning model comprise one or more of (i) a convolutional neural network including one or more convolutional blocks; (ii) a transformer neural network including one or more attention blocks; and (iii) a multi-layered perceptron neural network including one or more multi-layer perceptron blocks.

In some embodiments (FIG. 6F), the detail enhancement model is an ensemble network that includes at least a subset of a plurality of U-Net based neural networks and a plurality of generative adversarial network (GAN) based neural networks. In some embodiments, the intermediate layers of the detail enhancement model include one or more of a convolutional block, an attention block, and a multi-layer perceptron block.

In some embodiments, the detail enhancement model comprises a plurality of machine learning models (e.g., models 700 in FIGS. 7A-7D), and generating the second visual representation further includes applying each of the plurality of different machine learning models to process the first visual representation (e.g., successively, in parallel, etc.). For example, the detail enhancement model can include a series of different machine learning models (e.g., models 700A-700C in FIG. 7A), and generating the second visual representation further can include applying the series of machine learning models successively, wherein a first machine learning model (e.g., model 700A in FIG. 7A) is applied to process the first visual representation and generate a first intermediate representation (e.g., representation 712 in FIG. 7A, and a second machine learning model (e.g., model 700B in FIG. 7A) is applied to process the first intermediate representation and generate a second intermediate representation (e.g., representation 714 in FIG. 7A). In some embodiments (FIG. 7B or 7C), each of the plurality of different machine learning models corresponds to a respective intermediate visual representation generated based on the first visual representation, and the second visual representation is generated by averaging respective intermediate visual representations corresponding to the plurality of different machine learning models.

In some embodiments, a particular serial enhancement technique of the detail enhancement model includes: (i) training multiple machine learning models, each can individually increase optical textural details of axonal fiber bundles on ROTA map; (ii) applying one of the multiple trained models onto an input ROTA map, generating one version of enhanced ROTA map; and (iii) applying another one of the multiple models onto the enhanced ROTA map, repeating the process one-by-one for the remaining models.

In some embodiments (FIG. 7B), a particular parallel enhancement technique of the detail enhancement model includes: (i) training multiple machine learning models, each can individually increase optical textural details of axonal fiber bundles on ROTA map; (ii) applying the multiple trained machine learning models in parallel onto the input ROTA map, each model generating one version of enhanced ROTA map; (iii) overlaying the multiple enhanced ROTA maps generated from the multiple machine learning models; and (iv) averaging the values from multiple generated output images at each pixel location to one value to compose an averaged ROTA map.

In some embodiments, the detail enhancement model is deployed at a computing device, and the neural network of the detail enhancement model is trained at a server and provided to the computing device. That is, in some embodiments, the detail enhancement model, and/or constituent components thereof, are trained and/or deployed at a server, remote from the computer device. In some embodiments, the detail enhancement model is applied at a computing device, and the neural network of the detail enhancement model is trained locally at the computing device. In some embodiments, the computer system 300 deploys the detail enhancement model on a local computer or server in the same geometrical location as the eye being examined and scanned. In some embodiments, the computer system 300 deploys the detail enhancement model on a cloud computing platform where the input data (e.g., the first visual representation, which may be a ROTA map) is uploaded through internet.

In some embodiments, the detail enhancement model is configured to receive data corresponding to the first visual representation from a plurality of different OCT scanning machines. In some embodiments, data applied to the detail enhancement model includes a plurality of cross-sectional scan images of the retina captured by the plurality of OCT scanning machines, and are processed to generate the ROTA map, which is applied as the first visual representation.

In some embodiments, the detail enhancement model can be applied to one or more of the following: (i) a new ROTA map with low optical textural details of retinal axonal fiber bundles derived from OCT scans captured by the same model/variety of OCT imaging device for increasing the optical textural details; (ii) a new ROTA map with high optical textural details of retinal axonal fiber bundles derived from OCT scans captured by the same model/variety of OCT imaging device for further enhancing the optical textural details; (iii) a new ROTA map with low optical textural details of retinal axonal fiber bundles derived from OCT scans captured by a different model/variety of OCT imaging device for increasing optical textural details of the axonal fiber bundles; and/or (iv) a new ROTA map with high optical textural details of retinal axonal fiber bundles derived from OCT scans captured by the a different model/variety of OCT imaging device for further enhancing the optical textural details.

And the computer system 300, based on applying the detail enhancement model to the first visual representation, generates (850) a second visual representation of the retinal nerve fiber bundles of the retina, the second visual representation indicating the optical textural details with a second level of detail, distinct (e.g., greater than) from the first level of detail. For example, the output data 506 shown in FIG. 5 includes the second visual representation 410 which has distinct optical textural details from the visual representation 400. In some embodiments, the second visual representation has the same image resolution as the first visual representation, and the second level of detail includes a lower noise level or a smaller number of detectable features (e.g., features 404-408 in FIG. 4B) than the first level of detail. Alternatively, in some embodiments, the second visual representation has a larger image resolution as the first visual representation, resulting the second level of detail that is higher than the first level of detail.

In some embodiments, the second level of detail corresponds to one of two dimensions of the first visual representation, and the neural network of the detail enhancement model is applied to increase a resolution of the one of the two dimensions of the first visual representation. In some embodiments, the neural network of the detail enhancement model is applied to increase resolutions of both of the two dimensions of the first visual representation.

In some embodiments, the computer system 300 resizes (860) or sharpens the second visual representation. In some embodiments, the computer system 300 applies one or more applied machine learning models again to the second visual representation for further enhancement at least once. That is, in some embodiments, the second visual representation (e.g., an enhanced ROTA map) can be directly passed into the detail enhancement model again, or resampled to an image with lower resolution before passing into the model again.

In some embodiments, the computer system 300 determines (870) a health condition (e.g., a visual field sensitivity, a retinal nerve fiber defect) associated with the retina based on the second visual representation.

In some embodiments, the computer system 300 presents (880), at display of a computing device, the second visual representation of the retinal nerve fiber bundles. For example, FIGS. 4A to 4C show different visual representations of retinal nerve fiber bundles being displayed within the user interface 402. In some embodiments, alternatively or additionally to presentation the second visual representation, the computer system 300 presents, at the display of the computing device, a representation of a health condition of a patient (e.g., a field sensitivity representation) based on output data from the detail enhancement model.

In some embodiments, after the second visual representation is presented at the display, in accordance with receiving a user input requesting representation enhancement, the computer system 300 applies the detail enhancement model to the second visual representation to generate a third visual representation of the retinal nerve fiber bundles of the retina, and causes the third visual representation to be presented at the display of the computing device. In some embodiments, updated information about the patient's health condition is presented in conjunction with the third visual representation.

It should be understood that the particular order in which the operations in FIG. 8 have been described are merely exemplary and are not intended to indicate that the described order is the only order in which the operations could be performed. One of ordinary skill in the art would recognize numerous ways to apply a detail enhancement model to visual representations of optical textural details of a retina as described herein. Additionally, it should be noted that details of other processes described above with respect to FIGS. 1 to 7D are also applicable in an analogous manner to the method 800 described above with respect to FIG. 8. For brevity, many of these details are note repeated here.

Clause 1. A method of enhancing details of optical textural details of retinal features, comprising: obtaining a first visual representation of retinal nerve fiber bundles of a retina, the first visual representation indicating optical textural details of the retinal nerve fiber bundles of the retina with a first level of detail; applying a detail enhancement model to the first visual representation; and, based on applying the detail enhancement model to the first visual representation, generating a second visual representation of the retinal nerve fiber bundles of the retina, the second visual representation indicating the optical textural details with a second level of detail, distinct from the first level of detail.

Clause 2. The method of clause 1, further comprising determining a health condition associated with the retina based on the second visual representation.

Clause 3. The method of clause 1 or clause 2, further comprising: training a neural network of the detail enhancement model using a plurality of paired data samples, wherein each paired data sample includes a first respective visual representation and a second respective visual representation corresponding to a respective ground truth for a respective retina.

Clause 4. The method of clause 3, wherein a first respective data sample of the plurality of paired data samples includes the first respective visual representation of respective retinal nerve fiber bundles, the first respective visual representation having an input level of detail, and a second respective data sample of the plurality of paired data samples includes the second respective visual representation of the respective retinal nerve fiber bundles, the second respective visual representation having an output level of detail different than the input level of detail.

Clause 5. The method of clause 4, wherein: training the neural network of the detail enhancement model further includes obtaining the second respective visual representation of each of the paired data samples and down-sampling the second respective visual representation to generate the first visual representation; the first respective data sample includes one or more down-sampled images of the second respective data sample, and the one or more down-sampled images have a lower resolution than the second respective visual representation of the second respective data sample.

Clause 6. The method of any of clauses 3 to 5, wherein the second level of detail corresponds to one of two dimensions of the first visual representation, and the neural network of the detail enhancement model is applied to increase a resolution of the one of the two dimensions of the first visual representation.

Clause 8. The method of any of clauses 3 to 6, wherein the neural network of the detail enhancement model includes a first machine-learning model and a second machine-learning model, and the method further comprises: training the first machine-learning model using a first down-sampled set of images using a first sampling factor in first direction; and training the second machine-learning model using a second down-sampled set of images using a second sampling factor in a second direction.

Clause 8. The method of any of clauses 3 to 8, wherein an architecture of the neural network is based on: U-Net and includes an encoder and a decoder, and one or more layers of the encoder is coupled to one or more layers of the decoder via at least one skip connection or at least one cross connection, or GAN and comprises a generator sub-model and a discriminator sub-model.

Clause 9. The method of any of clauses 3 to 8, wherein the detail enhancement model is deployed at a computing device, and the neural network of the detail enhancement model is trained at a server and provided to the computing device.

Clause 10. The method of any of clauses 3 to 9, wherein the detail enhancement model is applied at a computing device, and the neural network of the detail enhancement model is trained locally at the computing device.

Clause 11. The method of any of clauses 1 to 10, wherein the retinal nerve fiber bundles of the retina include axonal fiber bundles of a retinal nerve fiber layer (RNFL) of the retina.

Clause 12. The method of any of clauses 1 to 11, wherein the first visual representation includes retinal optical texture analysis (ROTA) map of an inner retinal layer of the retina, the method further comprising: obtaining one or more cross-sectional scan images of the retina captured by an optical coherence tomography (OCT) device; and generating, using the one or more cross-sectional scan images of the retina, the ROTA map.

Clause 13. The method of clause 12, wherein: the ROTA map includes a plurality of pixels; and each pixel of the ROTA map includes a respective signature value S providing information about tissue composition and optical density of the inner retinal layer at a respective retinal location.

Clause 14. The method of clause 12 or clause 13, wherein obtaining the first visual representation includes identifying boundaries of a retinal nerve fiber layer (RNFL) based on one or more particular threshold transitions of optical density of the retinal nerve fiber bundles in the one or more cross-sectional scan images.

Clause 15. The method of any of clauses 12 to 14, wherein the ROTA map is pre-processed before being applied to the detail enhancement model.

Clause 16. The method of clause 15, wherein pre-processing the ROTA map includes one or more of: resizing to an image resolution that is more compatible with the detail enhancement model using one or more of nearest neighbor interpolation, bilinear interpolation, bicubic spline interpolation, and cubic spline interpolation; interlacing alternating blank horizontal lines between each vertical pair of neighbor rows of pixels of the ROTA map; interlacing alternating blank vertical lines between each horizontal pair of neighbor columns of the pixels of the ROTA map.

Clause 17. The method of clause 16, wherein pre-processing the ROTA map for training the detail enhancement model further includes one or more of: noisifying one or more respective pixels of the pixels of the ROTA map; blurring respective pixels of the ROTA map; and removing one or more respective pixels of the pixels of the ROTA map at each of one or more target locations.

Clause 18. The method of any of clauses 1 to 17, wherein the detail enhancement model is configured to receive data corresponding to the first visual representation from a plurality of different OCT scanning machines.

Clause 19. The method of any of clauses 1 to 18, wherein generating the second visual representation further comprises, after applying the detail enhancement model, resizing, or sharpening the second visual representation.

Clause 20. The method of any of clauses 1 to 19, further comprising presenting, at a display of a computing device, a representation of a health condition of a patient based on a patient health condition inferred from the optical textural details of the second visual representation.

Clause 21. The method of any of clauses 1 to 20, further comprising presenting, at a display of a computing device, the second visual representation of the retinal nerve fiber bundles of the retina.

Clause 22. The method of clause 21, further comprising, in accordance with receiving a user input requesting representation enhancement, applying the detail enhancement model to the second visual representation to generate a third visual representation of the retinal nerve fiber bundles of the retina; and causing the third visual representation to be presented at the display of the computing device.

Clause 23. The method of any of clauses 1 to 22, wherein the detail enhancement model is an ensemble network that includes at least a subset of a plurality of U-Net based neural networks and a plurality of generative adversarial network (GAN) based neural networks.

Clause 24. The method of any of clauses 1 to 23, wherein one or more intermediate layers of the detail enhancement model include one or more of a convolutional block, an attention block, and a multi-layer perceptron block.

Clause 25. The method of any of clauses 1 to 24, wherein the detail enhancement model comprises a plurality of different machine learning models; and generating the second visual representation further includes applying each of the plurality of different machine learning models to process the first visual representation.

Clause 26. The method of clause 25, wherein each of the plurality of different machine learning models corresponds to a respective intermediate visual representation generated based on the first visual representation; and the second visual representation is generated by averaging respective intermediate visual representations corresponding to the plurality of different machine learning models.

Clause 27. The method of any of clauses 1 to 26, wherein the detail enhancement model includes a series of different machine learning models; and generating the second visual representation further includes applying the series of machine learning models successively, wherein a first machine learning model is applied to process the first visual representation and generate a first intermediate representation, and a second machine learning model is applied to process the first intermediate representation and generate a second intermediate representation.

Clause 28. A non-transitory computer-readable storage medium comprising instructions that, when executed by one or more processors, cause the processors to perform the method of any of clauses 1 to 27.

Clause 29. A system, comprising: one or more processors, and memory, comprising instructions that, when executed by the one or more processors, cause the processors to perform the method of any of clauses 1 to 27.

In one or more examples, the functions described may be implemented in hardware, software, firmware, or any combination thereof. When implemented in software, the functions may be stored on or transmitted over, as one or more instructions or code, on a computer-readable medium, and executed by a hardware-based processing unit. Computer-readable media may include computer-readable storage media, which corresponds to a tangible medium such as a data storage medium, or communication media including any medium that facilitates transfer of a computer program from one place to another (e.g., according to a communication protocol). In this manner, computer-readable media generally may correspond to (1) tangible computer-readable storage media, which is non-transitory or (2) a communication medium such as a signal or carrier wave. Data storage media may be any available media that can be accessed by one or more computers or one or more processors to retrieve instructions, code and/or data structures for implementation of the embodiments described in the present application. A computer program product may include a computer-readable medium.

The terminology used in the description of the embodiments herein is for the purpose of describing particular embodiments only and is not intended to limit the scope of claims. As used in the description of the embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, elements, and/or components, but do not preclude the presence or addition of one or more other features, elements, components, and/or groups thereof.

It will also be understood that, although the terms first and second may be used herein to identify various elements, these elements should not be limited by these terms. These terms are only used to distinguish one element from another. For example, a first image could be termed a second image, and, similarly, a second image could be termed a first image, without departing from the scope of the embodiments. The first image and the second image are both images, but they are not the same image.

The description of the present application has been presented for purposes of illustration and description, and is not intended to be exhaustive or limited to the invention in the form disclosed. Many modifications, variations, and alternative embodiments will be apparent to those of ordinary skill in the art having the benefit of the teachings presented in the foregoing descriptions and the associated drawings. The embodiments were chosen and described in order to explain the principles of the invention, the practical applications, and to enable others skilled in the art to understand the invention for various embodiments and to utilize the underlying principles and various embodiments with various modifications as are suited to the particular use contemplated. Therefore, it is to be understood that the scope of claims is not to be limited to the specific examples of the embodiments disclosed and that modifications and other embodiments are intended to be included within the scope of the appended claims.

What is claimed is:

1. A method, comprising:
obtaining a first visual representation of retinal nerve fiber bundles of a retina, the first visual representation indicating optical textural details of the retinal nerve fiber bundles of the retina with a first level of detail;
applying a detail enhancement model to the first visual representation;
based on applying the detail enhancement model to the first visual representation, generating a second visual representation of the retinal nerve fiber bundles of the retina, the second visual representation indicating the optical textural details with a second level of detail, distinct from the first level of detail; and
determining a health condition associated with the retina based on the second visual representation.

2. The method of claim 1, further comprising:
training a neural network of the detail enhancement model using a plurality of paired data samples, wherein each paired data sample includes a first respective visual representation and a second respective visual representation corresponding to a respective ground truth for a respective retina.

3. The method of claim 2, wherein:
a first respective data sample of the plurality of paired data samples includes the first respective visual representation of respective retinal nerve fiber bundles, the first respective visual representation having an input level of details, and
a second respective data sample of the plurality of paired data samples includes the second respective visual representation of the respective retinal nerve fiber bundles, the second respective visual representation having an output level of details different than the input level of details.

4. The method of claim 3, wherein:
training the neural network of the detail enhancement model further includes obtaining the second respective visual representation of each of the paired data samples and down-sampling the second respective visual representation to generate the first visual representation;
the first respective data sample includes one or more down-sampled images of the second respective data sample; and
the one or more down-sampled images have a lower resolution than the second respective visual representation of the second respective data sample.

5. The method of claim 2, wherein the second level of details corresponds to one or more of two dimensions of the first visual representation, and the neural network of the detail enhancement model is applied to increase a resolution of the one or more of the two dimensions of the first visual representation.

6. The method of claim 2, wherein the neural network of the detail enhancement model includes a first machine-learning model and a second machine-learning model, and the method further comprises:
training the first machine-learning model using a first down-sampled set of images using a first sampling factor in a first direction; and
training the second machine-learning model using a second down-sampled set of images using a second sampling factor in a second direction.

7. The method of claim 2, wherein an architecture of the neural network is based on:
U-Net and includes an encoder and a decoder, and one or more layers of the encoder is coupled to one or more layers of the decoder via at least one skip connection or at least one cross connection, or GAN and comprises a generator sub-model and a discriminator sub-model.

8. The method of claim 1, wherein the first visual representation includes retinal optical texture analysis (ROTA) map of an inner retinal layer of the retina, the method further comprising:
obtaining one or more cross-sectional scan images of the retina captured by an optical coherence tomography (OCT) device; and
generating, using the one or more cross-sectional scan images of the retina, the ROTA map which includes a plurality of pixels and each pixel of the ROTA map includes a respective signature value S providing information about tissue composition and optical density of the inner retinal layer at a respective retinal location.

9. The method of claim 8, wherein the ROTA map is pre-processed before being received by the detail enhancement model.

10. The method of claim 9, wherein pre-processing the ROTA map includes one or more of:
resizing to an image resolution that is more compatible with the detail enhancement model using one or more of nearest neighbor interpolation, bilinear interpolation, bicubic spline interpolation, and cubic spline interpolation;
interlacing alternating blank horizontal lines between each vertical pair of neighbor rows of pixels of the ROTA map; and
interlacing alternating blank vertical lines between each horizontal pair of neighbor columns of the pixels of the ROTA map.

11. The method of claim 9, wherein pre-processing the ROTA map for training the detail enhancement model further includes one or more of:
noisifying one or more respective pixels of the pixels of the ROTA map;
blurring respective pixels of the ROTA map; and
removing one or more respective pixels of the pixels of the ROTA map at each of one or more target locations.

12. The method of claim 1, wherein the trained detail enhancement model is configured to receive data corresponding to the first visual representation from a plurality of different OCT scanning machines.

13. The method of claim 1, wherein generating the second visual representation further comprises:
after applying the detail enhancement model, resizing, or sharpening the second visual representation.

14. The method of claim 1, further comprising one or more of:
presenting, at a display of a computing device, the second visual representation of the retinal nerve fiber bundles of the retina; and
presenting, at a display of a computing device, a representation of a health condition of a patient based on inferring the health condition from the optical textural details of the second visual representation.

15. The method of claim 1, wherein the detail enhancement model is an ensemble network that includes at least a subset of a plurality of U-Net based neural networks and a plurality of generative adversarial network (GAN) based neural networks.

16. The method of claim 1, wherein:
the detail enhancement model comprises a plurality of different machine learning models; and
generating the second visual representation further includes applying each of the plurality of different machine learning models to process the first visual representation, with each of the plurality of different machine learning models corresponds to a respective intermediate visual representation generated based on the first visual representation, and the second visual representation is generated by averaging respective intermediate visual representations corresponding to the plurality of different machine learning models.

17. The method of claim 1, wherein:
the detail enhancement model includes a series of different machine learning models; and
generating the second visual representation further includes applying the series of machine learning models successively, wherein a first machine learning model is applied to process the first visual representation and generate a first intermediate representation, and a second machine learning model is applied to process the first intermediate representation and generate a second intermediate representation.

18. The method of claim 1, wherein the health condition associated with the retina to be determined is a presence of a defect in a retinal nerve fiber layer (RNFL) of the retina.

19. A non-transitory computer-readable storage medium comprising instructions that, when executed by one or more processors, cause the processors to:
obtain a first visual representation of retinal nerve fiber bundles of a retina, the first visual representation indicating optical textural details of the retinal nerve fiber bundles of the retina with a first level of detail;
apply a detail enhancement model to the first visual representation;
based on applying the detail enhancement model to the first visual representation, generating a second visual representation of the retinal nerve fiber bundles of the retina, the second visual representation indicating the optical textural details with a second level of detail, distinct from the first level of detail; and
determine a health condition associated with the retina based on the second visual representation.

20. A system, comprising:
one or more processors, and
memory, comprising instructions that, when executed by the one or more processors, cause the processors to:
obtain a first visual representation of retinal nerve fiber bundles of a retina, the first visual representation indicating optical textural details of the retinal nerve fiber bundles of the retina with a first level of detail;
apply a detail enhancement model to the first visual representation; and
based on applying the detail enhancement model to the first visual representation, generating a second visual representation of the retinal nerve fiber bundles of the retina, the second visual representation indicating the optical textural details with a second level of detail, distinct from the first level of detail; and
determine a health condition associated with the retina based on the second visual representation.
